(19) United States
(12) Patent Application Publication
(10) Pub. No.: US 2022/0401533 A1
(43) Pub. Date: Dec. 22, 2022

(54) TREATMENT AND PROTECTION AGAINST ASPERGILLUS INFECTION AND ASPERGILLOSIS DISEASE

(71) Applicant: University of Georgia Research Foundation, Inc., Athens, GA (US)

(72) Inventors: Emily Anne RAYENS, Athens, GA (US); Whitney RABACAL, Athens, GA (US); Karen NORRIS, Athens, GA (US)

(73) Assignee: University of Georgia Research Foundation, Inc., Athens, GA (US)

(21) Appl. No.: 17/776,938

(22) PCT Filed: Nov. 12, 2020
(86) PCT No.: PCT/US2020/060125

§ 371 Date: May 13, 2022

Related U.S. Application Data
(60) Provisional application No. 62/935,280, filed on Nov. 14, 2019.

(51) Int. Cl. A61K 39/00; C07K 16/14; C07K 14/38; A61P 31/10

(52) U.S. Cl. CPC A61K 39/0002; C07K 16/14; C07K 14/38; A61P 31/10

(57) ABSTRACT

The invention generally provides methods of treating or preventing aspergillosis disease and/or its symptoms associated with infection by the Aspergillus pathogenic fungus. The methods involve administering an Aspergillus Kexin peptide, or a composition comprising an Aspergillus peptide, to a mammalian subject in need thereof, such as a subject afflicted with aspergillus, or a subject susceptible to or at risk of infection by Aspergillus and ensuing aspergillosis disease. In some aspects, the Aspergillus Kexin peptide is an A. fumigatus Kexin peptide. In some aspects, the mammalian subject is a human patient. In some aspects, the patient is immunosuppressed or immunocompromised. The Aspergillus Kexin peptide as immunogen or vaccine generates a potent and robust immune response, e.g., antibody response, in the immunized subject. The methods afford therapeutic and protective treatment against aspergillosis and its symptoms, as well as a reduction in the severity of aspergillosis in the treated subjects.

### CROSS-REFERENCE TO RELATED APPLICATIONS

This application claims priority to and benefit of U.S. Provisional Application No. 62/935,280, filed Nov. 14, 2019, the entire contents of which are incorporated herein by reference.

### BACKGROUND

Infection with the opportunistic fungal pathogen Aspergillus, e.g., Aspergillus fumigatus, causes severe invasive pulmonary disease, particularly, in immunocompromised individuals. Those at risk for Aspergillus infection include individuals with neutropenia caused by immunosuppressive therapies associated with transplantation, cancer, or autoimmune disease. Aspergillosis is a disease caused by Aspergillus infection in a subject.

The rise in the use of chemotherapy and immunosuppressive agents has increased the incidence of invasive pulmonary aspergillosis (IPA). Antifungal treatment of this disease is not always successful, as evidenced by the mortality rate of IPA, which continues to exceed 50% in neutropenic patients. Moreover, treatment does not prevent future infection in continually susceptible patients, and there are no clinically-approved vaccines to prevent opportunistic fungal infections. Therefore, a serious and unmet need exists for immunogens and vaccines that are effective in treating and protecting against infection and aspergillosis disease caused by Aspergillus, including IPA.

### SUMMARY

Provided herein are methods for treating disease or protecting a subject from developing disease and/or the symptoms thereof caused by the fungal pathogen Aspergillus. In an embodiment, the subject is infected by, is susceptible to or is at risk of infection by an Aspergillus pathogen. In embodiments, the disease is pulmonary disease and poor pulmonary function associated with Aspergillus infection. In a particular embodiment, the disease is aspergillosis. In an embodiment, infection and aspergillosis disease are caused by the fungal pathogen Aspergillus fumigatus (A. fumigatus).

In various aspects, the methods described and exemplified herein relate to the finding that a mammalian subject, immunized (e.g., inoculated) with a Kexin peptide (e.g., AF.KEX1 peptide described herein) derived from the Kexin (KEX) protein (polypeptide), a subtilisin-like serine protease of an Aspergillus fumigatus fungal pathogen, elicited an immune response, e.g., high titer anti-A. fumigatus KEX peptide antibodies, that provided immunoprotection in the immunized subject against aspergillosis disease following challenge and infection with Aspergillus fungal organisms. Immunoprotection in the subject included the elicitation of an immune response against the Aspergillus Kexin (KEX) peptide as immunogen. In embodiments, the immune response involves the elicitation of a robust humoral (e.g., anti-Aspergillus Kexin peptide antibody production by B cells) response and a cellular (activated T cells) response. In an embodiment, the Aspergillus fungal organism is Aspergillus fumigatus. In an embodiment, the Aspergillus Kexin peptide is derived from the Kexin protein of Aspergillus fumigatus, e.g., an A. fumigatus KEX peptide, such as AF.KEX1 as described herein. In an embodiment, the subject is immunosuppressed or immunocompromised. In an embodiment, a mammalian subject immunized with an A. fumigatus KEX peptide, e.g., the AF.KEX1 peptide, as immunogen was protected from developing aspergillosis disease and survived A. fumigatus infection for a longer time period compared with an unimmunized (sham treated) control mammalian subject. In an embodiment, the subject is a human patient.

It will be understood that the term “Kexin” protein or peptide is used interchangeably herein with the abbreviated term “KEX” protein or peptide.

In an aspect, a method of treating a subject having aspergillosis disease and/or the symptoms thereof is provided, in which the method involves administering to a subject in need thereof an effective amount of a Kexin peptide derived from an Aspergillus fungal pathogen to treat aspergillosis disease and/or the symptoms thereof in the subject.

In an aspect, a method of protecting a subject against aspergillosis disease and/or the symptoms thereof is provided, in which the method involves administering to a subject in need thereof an effective amount of a Kexin peptide derived from an Aspergillus fungal pathogen to protect the subject against aspergillosis disease and/or the symptoms thereof. In an embodiment, the subject is susceptible to or is at risk of infection by an Aspergillus fungal pathogen.

In an aspect, a method of reducing the severity of aspergillosis disease and/or the symptoms thereof in a subject is provided, in which the method involves administering to a subject in need thereof an effective amount of a Kexin peptide derived from an Aspergillus fungal pathogen to reduce the severity of aspergillosis disease and/or the symptoms thereof in the subject. In an embodiment, the subject is susceptible to or is at risk of infection by an Aspergillus fungal pathogen.

In embodiments of the methods of any of the foregoing delineated aspects, the Aspergillus Kexin peptide is selected from a 90-amino acid peptide comprising or consisting of SEQ ID NO: 2 (referred to as AF.KEX1 herein) or an 88-amino acid A. fumigatus KEX peptide comprising or consisting of SEQ ID NO: 3. In embodiments, the 90-amino acid AF.KEX1 peptide or the 88-amino acid A. fumigatus KEX peptide is encoded by a polynucleotide contained in an expression vector. In embodiments of the methods, the Aspergillus fungal pathogen is selected from Aspergillus fumigatus (A. fumigatus), Aspergillus flavus (A. flavus), Aspergillus terreus (A. terreus), Aspergillus nidulans (A. nidulans), Aspergillus versicolor (A. versicolor), or Aspergillus niger (A. niger). In a particular embodiment, the Aspergillus fungal pathogen is Aspergillus fumigatus (A. fumigatus). In embodiments of the methods, the aspergillosis disease is selected from allergic bronchopulmonary aspergillosis (ABPA), allergic Aspergillus sinusitis, aspergilloma, chronic pulmonary aspergillosis, invasive pulmonary aspergillosis (IPA), or cutaneous aspergillosis. In a particular embodiment, the aspergillosis disease is invasive pulmonary aspergillosis (IPA). In an embodiment of the methods, the subject is immunosuppressed or immunocompromised. In an embodiment, the subject is human.

In another aspect, a method of preventing or reducing the development of aspergillosis associated with infection by an Aspergillus fungal organism is provided, in which the method involves administering to a subject in need thereof an Aspergillus Kexin peptide in an amount effective to elicit an immune response comprising Aspergillus Kexin peptide-specific antibodies in the subject, wherein the anti-Aspergillus Kexin peptide antibodies prevent or reduce the development of aspergillosis associated with infection by the Aspergillus fungal organism in the subject.

In another aspect, a method of reducing lung fungal burden associated with infection by an Aspergillus fungal organism is provided, in which the method involves administering to a subject in need thereof an Aspergillus Kexin peptide in an amount effective to elicit an immune response comprising Aspergillus Kexin peptide-specific antibodies in the subject, wherein the anti-Aspergillus Kexin peptide antibodies reduce lung fungal burden associated with infection by the Aspergillus fungal organism in the subject.

In another aspect, a method of eliciting a humoral immune response that is immunoprotective against aspergillosis and/or the symptoms thereof associated with infection by an Aspergillus fungal organism is provided, in which the method involves administering to a subject in need thereof an Aspergillus Kexin peptide in an amount effective to elicit a humoral immune response comprising Aspergillus Kexin peptide-specific antibodies in the subject, wherein the humoral immune response immunoprotects the subject against aspergillosis and/or the symptoms thereof associated with infection by the Aspergillus fungal organism.

In embodiments of the methods of any of the foregoing delineated aspects, the titer of the antibodies generated against the Aspergillus Kexin peptide is inversely correlated with Aspergillus fungal burden in lung. In an embodiment, the Aspergillus Kexin peptide is an Aspergillus fumigatus (A. fumigatus) Kexin peptide. In an embodiment, the Aspergillus Kexin peptide is selected from a 90-amino acid peptide comprising or consisting of SEQ ID NO: 2 or an 88-amino acid peptide comprising or consisting of SEQ ID NO: 3. In an embodiment, the 90-amino acid Aspergillus Kexin peptide or the 88-amino acid Aspergillus Kexin peptide is encoded by a polynucleotide contained in an expression vector. In a particular embodiment, the A. fumigatus KEX peptide is a 90-amino acid peptide comprising or consisting of SEQ ID NO: 2 (AF.KEX1). In an embodiment, the Aspergillus fungal organism is selected from Aspergillus fumigatus (A. fumigatus), Aspergillus flavus (A. flavus), Aspergillus terreus (A. terreus), Aspergillus nidulans (A. nidulans), Aspergillus versicolor (A. versicolor), or Aspergillus niger (A. niger). In a particular embodiment, the Aspergillus fungal organism is Aspergillus fumigatus (A. fumigatus). In an embodiment, the aspergillosis disease is selected from allergic bronchopulmonary aspergillosis (ABPA), allergic Aspergillus sinusitis, aspergilloma, chronic pulmonary aspergillosis, invasive pulmonary aspergillosis (IPA), or cutaneous aspergillosis. In a particular embodiment, the aspergillosis disease is invasive pulmonary aspergillosis (IPA). In an embodiment, the subject is human. In an embodiment, the subject is immunosuppressed or immunocompromised.

In another aspect, a method of treating or protecting an immunosuppressed patient against developing aspergillosis and/or the symptoms thereof associated with infection by an Aspergillus fungal pathogen is provided, in which the method involves administering to a patient who is to receive, is receiving, or has received an immune suppressive drug, agent, or medication a Kexin peptide derived from an Aspergillus fungal pathogen in an amount effective for the patient to generate anti Aspergillus Kexin peptide antibodies and acquire protective immunity to treat or protect the immunosuppressed patient against developing aspergillosis and/or the symptoms thereof. In an embodiment of the method, the Kexin peptide immunogen derived from an Aspergillus fumigatus fungal pathogen is administered to the patient.

In another aspect, a method of treating or protecting an immunosuppressed patient against developing aspergillosis and/or the symptoms thereof associated with infection by an Aspergillus fungal pathogen is provided, in which the method involves administering to a patient who is to receive, is receiving, or has received an immune suppressive drug or medication an isolated antiserum comprising an antibody produced against the Aspergillus Kexin peptide, or an isolated and purified antibody produced against the Aspergillus Kexin peptide, in an amount effective for the patient to acquire protective immunity to treat or protect the immunosuppressed patient against developing aspergillosis and/or the symptoms thereof.

In embodiments of the methods of any of the foregoing delineated aspects, the patient has congenital or acquired immunosuppression, is undergoing treatment with an immunosuppressive drug, agent, or medicament, is undergoing treatment with an anticancer, chemotherapeutic, anti-inflammatory or immuno-oncology drug, agent, or medicament, or is a pre-transplant patient or a post-transplant patient. In an embodiment, the patient is to receive, is receiving, or has received one or more immunosuppressive drugs or agents. In accordance with the methods of any of the foregoing delineated aspects, a patient may be immunosuppressed or immunocompromised from different causes, for example, congenital immunosuppression and/or the treatment thereof, or treatment of a medical condition, disease, illness, or pathology with an immunosuppressive drug or agent, e.g., drug induced immunosuppression. By way of nonlimiting example, an immunosuppressed patient may have a disease, condition, or pathology, which results in immunosuppression, or for which the patient is administered an immunosuppressive drug, agent, or treatment, e.g., chemotherapy or immunotherapy. In embodiments, the disease, condition, or pathology is, without limitation, an inflammatory disease, systemic lupus erythematosus, rheumatoid arthritis, irritable bowel disease, psoriasis, eczema, Crohn's disease, or cancer. In an embodiment, the patient is to receive, is receiving, or has received one or more immunosuppressive drugs or agents as described herein. In an embodiment, the patient is a pre-transplant patient or a post-transplant patient. In an embodiment, In an embodiment, the Aspergillus fungal pathogen is selected from Aspergillus fumigatus (A. fumigatus), Aspergillus flavus (A. flavus), Aspergillus terreus (A. terreus), Aspergillus nidulans (A. nidulans), Aspergillus versicolor (A. versicolor), or Aspergillus niger (A. niger). In a particular embodiment, the Aspergillus fungal pathogen is Aspergillus fumigatus (A. fumigatus). In an embodiment, the aspergillosis is selected from allergic bronchopulmonary aspergillosis (ABPA), allergic Aspergillus sinusitis, aspergilloma, chronic pulmonary aspergillosis, invasive pulmonary aspergillosis (IPA), or cutaneous aspergillosis. In a particular embodiment, the aspergillosis is invasive pulmonary aspergillosis (IPA).

In yet another aspect, a method of treating or protecting a subject from aspergillosis and/or the symptoms thereof associated with infection by an Aspergillus fungal pathogen is provided, in which the method involves administering to a subject in need thereof an isolated antiserum comprising an antibody directed against an Aspergillus Kexin peptide immunogen, or an isolated and purified antibody directed against the Aspergillus Kexin peptide immunogen, in an amount effective to treat or protect the subject from aspergillosis and/or the symptoms thereof. In an embodiment, the antiserum comprises a monoclonal antibody, a polyclonal antibody, or a combination thereof. In an embodiment, the antiserum or the antibody is generated against a 90-amino acid A. fumigatus Kexin peptide comprising or consisting of SEQ ID NO: 2 (AF.KEX1). In an embodiment, the antiserum or the antibody is generated against an 88-amino acid A. fumigatus Kexin peptide comprising or consisting of SEQ ID NO: 3. In an embodiment, the subject has or is at risk of having infection or disease caused by an Aspergillus fungal pathogen. In an embodiment, the subject is immunocompromised or immunosuppressed. In an embodiment, the Aspergillus fungal pathogen or the Aspergillus Kexin peptide immunogen is selected from Aspergillus fumigatus (A. fumigatus), Aspergillus flavus (A. flavus), Aspergillus terreus (A. terreus), Aspergillus nidulans (A. nidulans), Aspergillus versicolor (A. versicolor), or Aspergillus niger (A. niger) fungal pathogen or Kexin peptide immunogen. In a particular embodiment, the Aspergillus fungal pathogen is an Aspergillus fumigatus (A. fumigatus) fungal pathogen or the Aspergillus Kexin peptide immunogen is an A. fumigatus Kexin peptide immunogen. In an embodiment, the aspergillosis is selected from allergic bronchopulmonary aspergillosis (ABPA), allergic Aspergillus sinusitis, aspergilloma, chronic pulmonary aspergillosis, invasive pulmonary aspergillosis (IPA), or cutaneous aspergillosis. In a particular embodiment, the aspergillosis is invasive pulmonary aspergillosis (IPA). In an embodiment, the subject is human.

In an embodiment of the methods of any of the foregoing delineated aspects, the methods further involve treating or protecting the subject or patient against colonization of the Aspergillus fungal pathogen or organism in lung tissue. In an embodiment of the methods of any of the foregoing delineated aspects, the methods further involve treating or protecting the subject or patient against aspergilloma associated with infection by the Aspergillus fungal pathogen or organism. In an embodiment of the methods of any of the foregoing delineated aspects, mortality associated with Aspergillus infection and/or aspergillosis disease is reduced in the subject or patient. In an embodiment of the methods of any of the foregoing delineated aspects, Aspergillus fungal burden in lung associated with Aspergillus infection and/or aspergillosis disease is reduced in the subject or patient. In an embodiment of the methods of any of the foregoing delineated aspects, the Aspergillus Kexin peptide is administered with an adjuvant. In an embodiment of the methods of any of the foregoing delineated aspects, the Aspergillus Kexin peptide is administered in a pharmaceutically acceptable composition. In an embodiment of the methods of any of the foregoing delineated aspects, the Aspergillus Kexin peptide or antiserum is administered in conjunction with another therapeutic agent or treatment.

In another aspect is provided a kit comprising an Aspergillus Kexin peptide of SEQ ID NO: 2 or an Aspergillus Kexin peptide of SEQ ID NO: 3, an expression vector comprising a polynucleotide encoding the Aspergillus Kexin peptide, or an isolated antiserum comprising antibodies specifically directed against Aspergillus Kexin peptide for use in the methods of any one of the methods of the foregoing delineated aspects.

In other aspects, antiserum obtained or isolated from a subject immunized with A. fumigatus KEX peptide (e.g., AF.KEX1) contains anti-A. fumigatus KEX peptide antibodies that are immunoprotective against A. fumigatus infection and aspergillosis associated with A. fumigatus infection. Such antiserum can serve as a therapeutic or preventative treatment for A. fumigatus infection and/or aspergillosis disease and can provide immunity against A. fumigatus and aspergillosis in another or unrelated subject (i.e., a recipient subject) who receives the antiserum via a suitable mode and route of administration. It will be appreciated by the skilled practitioner that, as used herein, a subject from whom an isolated antiserum is obtained is a “donor subject,” and a subject to whom the isolated antiserum is administered or provided is a “recipient subject.” In embodiments, the subject is a mammal, particularly a human being or a non-human primate. A recipient subject may be a patient or an individual in need of treatment for or protection from Aspergillus infection and aspergillosis disease. In an embodiment, the antiserum is an isolated antiserum. In embodiments, the isolated antiserum may be processed, e.g., concentrated, diluted in a suitable diluent or excipient, chromatographed, purified, e.g., via affinity chromatography, using procedures practiced by one of skill in the art, prior to its use as a therapeutic in the methods described herein. In an embodiment, the anti-Aspergillus KEX peptide antibodies isolated (and/or purified) from the immunized subject are monoclonal antibodies (e.g., specifically directed against the KEX peptide of Aspergillus. In an embodiment, the anti-Aspergillus KEX peptide antibodies isolated (and/or purified) from the immunized subject are polyclonal antibodies (e.g., specifically directed against the KEX protein or peptide of Aspergillus. In an embodiment, the isolated antiserum is administered in a pharmaceutically acceptable composition.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By a “Kexin,” “KEX,” or “KEX1” protein is meant a polypeptide or peptide fragment thereof having at least about 85% or greater, about 90% or greater, about 91, 92, 93, 94, 95, 96, 97, 98, 99% or greater amino acid identity to the amino acid sequence of an exemplary KEX (KEXB endoprotease) polypeptide sequence of Aspergillus fumigatus (Af293) having NCBI Accession No. XM_746441.1 is provided below:

(SEQ ID NO: 1)
MRFLGSIALVLSSISVASANVRSRSYDTHEFFALHLDDSASPSHVAQLLG
ARHEGQIGELANHHTFSIPRERSSDLDALLERARAARKIRRRARDDATSQ
EQHNDALGGILWSQKLAPKKRLVKRVPPPERLARTFATGKEDPVAAQSQK
RIASTLGITDPIFNGQWHLFNTVQLGHDLNVTGVWMEGITGKGVTTAVVD
DGLDMYSNDLKPNYFPEGSYDFNDHTPEPRPRLSDDKHGTRCAGEIAAAR
NDVCGVGVAYDSRVAGVRILSKAIDDADEATAINFAYQENDIFSCSWGPP
DDGATMEGPGILIKRAFVNGVQNGRGGKGSIFVFAAGNGASFEDNCNFDG
YTNSIYSITVGAIDREGNHPSYSESCSAQLVVAYSSGSGDAIHTTDVGTD
KCYSFHGGTSAAGPLAAGTVALALSARPELTWRDAQYLMVETAVPIHEDD
GSWQVTKAGRKFSHDWGYGKVDAYALVQKAKTWELVKPQAWFHSPWLRVQ
HKVPQGDQGLASSYEVTEQMMKNANIARLEHVTVTMNVNHTRRGDLSVEL
RSPEGIVSHLSTTRKSDNEKAGYVDWTFMTVAHWGESGVGRWTVIVKDTN
VNEFTGEFIDWRLNLWGEAIDGANQKPHPFPDEHDDDHSIEDAIVATTSV
ETGPTKTGVPGSTDDTINRPVNAKPVETQTPSPAETTATKLAPPAETRPA
ATATSSPTPPAASDSFLPSFMPTFGASKRTQIWIYAAIGSIIVFCIGLGI
YFQVQRRKRILNNPRDDYDFEMIEDENALHGGNGRSGRTQRRGGELYNAF
AGESDEEEPLFSDEDDEPYRDRAPSEDRLRDTSSDDRSLRHGDH

Shown below is the amino acid sequence of a 90-amino acid region (“90-mer”) of the Aspergillus fumigatus Kexin protein, a subtilisin-like serine protease. The 90-amino acid region is an Aspergillus fumigatus Kexin peptide, also called (A. fumigatus KEX1 or AF.KEX1 peptide herein).

(SEQ ID NO: 2)

| | |
|---|---|
| 1 | PDDGATMEGP GILIKRAFVN GVQNGRGGKG SIFVFAAGNG |
| | ASFEDNCNFD |
| 51 | GYTNSIYSIT VGAIDREGNH PSYSESCSAQ LVVAYSSGSG. |

In an embodiment, the Aspergillus KEX1 peptide constitutes the pheromone processing endoprotease KEXB of A. fumigatus strain Af293 encoded by polynucleotide AFUA_4G12970 under UniProtKB Accession No. Q4WQ18_ASPFU. In an embodiment, a polynucleotide encoding the 90-mer AF.KEX1 peptide is contained in an expression vector construct.

Shown below is the amino acid sequence of an 88-amino acid region (“88-mer”) of the Aspergillus fumigatus Kexin protein, a subtilisin-like serine protease. The 88-amino acid region is an Aspergillus fumigatus Kexin peptide, which is also referred to as A. fumigatus KEX peptide herein.

(SEQ ID NO: 3)

| | |
|---|---|
| 1 | DDGATMEGPG ILIKRAFVNG VQNGRGGKGS IFVFAAGNGA |
| | SFEDNCNFDG |
| 51 | YTNSIYSITV GAIDREGNHP SYSESCSAQL VVAYSSGS. |

In an embodiment, a polynucleotide encoding the 88-mer Aspergillus KEX peptide is contained in an expression vector construct.

As used herein, the term “AF.KEX1” peptide refers to an A. fumigatus Kexin peptide comprising or consisting of SEQ ID NO: 2. In an embodiment, the AF.KEX1 peptide is used as an immunogen as described and exemplified herein. In another embodiment, an A. fumigatus KEX peptide comprises or consists of SEQ ID NO: 3. In an embodiment, the A. fumigatus KEX peptide comprising or consisting of SEQ ID NO: 3 is used as an immunogen.

By “agent” is meant a peptide, nucleic acid molecule, or small compound, or an immunogen comprising one or more of the foregoing.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

The term “antibody,” as used herein, refers to an immunoglobulin molecule which specifically binds with an antigen. Methods of preparing antibodies are well known to those of ordinary skill in the science of immunology. Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. Antibodies also include dimers that may be naturally occurring or constructed from single chain antibodies or antibody fragments. The antibodies in the present invention may exist in a variety of forms including, for example, polyclonal antibodies, monoclonal antibodies, Fv, Fab and F(ab′) 2, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′) 2, and Fv fragments, linear antibodies, scFv antibodies, single-domain antibodies, such as camelid antibodies (Riechmann, 1999, Journal of Immunological Methods, 231:25-38), composed of either a VL or a VH domain which exhibit sufficient affinity for the target, and multispecific antibodies formed from antibody fragments. The antibody fragment also includes a human antibody or a humanized antibody or a portion of a human antibody or a humanized antibody.

Antibodies can be made by any of the methods known in the art utilizing a polypeptide (e.g., a Kexin polypeptide), or immunogenic peptide fragments thereof, as an immunogen. One method of obtaining antibodies is to immunize suitable host animals with an immunogen and to follow standard procedures for polyclonal or monoclonal antibody production. The immunogen facilitates the presentation of the immunogenic fragments on the cell surface. Immunization of a suitable host can be carried out in several ways. Nucleic acid sequences encoding a polypeptide of the invention, or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding the polypeptide, or immunogenic fragments thereof, can be expressed in cells in vitro, followed by isolation of the polypeptide and administration of the polypeptide to a suitable host in which antibodies are raised.

Alternatively, antibodies against the polypeptide may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

By “anti-Kexin protein antibody,” or “anti-KEX peptide antibody,” is meant an antibody or an antigen binding fragment thereof that selectively binds a Kexin polypeptide or a peptide fragment thereof, including, for example, AF.KEX1, a peptide derived from the Kexin polypeptide of the Aspergillus fumigatus fungal pathogen, as described herein. In specific embodiments, the anti-KEX peptide antibody specifically binds a binding site of the Kexin protein or KEX peptide of an Aspergillus fungal pathogen, such as Aspergillus fumigatus. In an embodiment, the Aspergillus fumigatus KEX peptide is a 90-mer amino acid sequence (SEQ ID NO: 2), (AF.KEX1 peptide). In an embodiment, the Aspergillus fumigatus KEX peptide is an 88-mer amino acid sequence (SEQ ID NO: 3).

An “antiserum” refers to blood serum that contains one or more antibodies directed against a specific antigen. Antiserum containing antibodies may be obtained from the blood or serum of an animal (a mammal), including a human, that has been immunized or inoculated with an immunogen (or an antigen material) either by injection, typically into the bloodstream or tissues, or by infection. In an embodiment, the animal (a mammal), including a human, may be immunized or inoculated with the blood or serum of an organism or individual whose immune system has been stimulated to generate an immune response (e.g., antibody production) by infection or natural contact with an antigenic material or immunogen. In this case, an antiserum contains anti-KEX peptide antibodies, e.g., polyclonal antibodies or populations of monoclonal antibodies, generated or produced by an immunized, inoculated, or exposed donor subject against a KEX peptide immunogen derived from an Aspergillus fungal pathogen, such as Aspergillus fumigatus. Such antiserum, isolated (and/or purified) from the donor subject can be used to immunize (i.e., administer to) another (unrelated) subject so as to provide immunity (acquired immunity) against infection or disease caused by or associated with an Aspergillus fungal pathogen, such as Aspergillus fumigatus. In this way, a subject who receives the antiserum, i.e., antibodies in the antiserum, is passively treated or protected against infection and/or disease caused by an Aspergillus fungal pathogen, such as Aspergillus fumigatus. Such antiserum-derived immunoprotection against an Aspergillus fungal pathogen, such as Aspergillus fumigatus, constitutes an acquired or passive immunity obtained by the recipient subject and imparted from the donor subject's isolated antiserum. As will be appreciated by one skilled in the art, blood serum is the amber-colored, protein-rich liquid component of blood that separates from the clot when blood coagulates. The serum component containing one or more antibodies (cross-protective antibodies) is termed “antiserum.” In an embodiment, the antiserum is an isolated antiserum, e.g., isolated from a donor subject. In an embodiment, an isolated antiserum may be processed by methods used by one skilled in the art, such as dilution, concentration (e.g., via filtration or centrifugation or both), chromatography, purification to remove ions or extraneous protein, and the like, prior to its use as a treatment or protective therapeutic as described herein. In an embodiment, an isolated antiserum may be further purified after isolation. In an embodiment, an isolated antiserum is not further processed or purified. In an embodiment, antibodies, or antigen-binding fragments thereof, contained in an isolated antiserum may be further isolated by methods practiced by those having skill in the art, such as, without limitation, by affinity chromatography, size exclusion chromatography, immunoprecipitation, dialysis, HPLC chromatography, etc.

By “biological sample” is meant any liquid, cell, or tissue obtained from a subject. In some embodiments, the biological sample is blood, serum, plasma, cerebrospinal fluid (CFS), bronchoalveolar lavage, pulmonary lavage, sputum, tears, saliva, urine, semen, feces, etc.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte that is detected or that is to be detected.

By “disease” is meant any condition, dysfunction, or disorder that damages or interferes with the normal function of a cell, tissue, or organ. In an embodiment, the disease is aspergillosis. In embodiments, the aspergillosis disease includes allergic bronchopulmonary aspergillosis (ABPA), allergic Aspergillus sinusitis, aspergilloma, chronic pulmonary aspergillosis, invasive pulmonary aspergillosis (IPA), or cutaneous aspergillosis. In other embodiments, the disease is a pulmonary (lung) disease. In a particular embodiment, the aspergillosis disease is IPA.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. An immunologically effective amount of an isolated antiserum of the invention is an amount required to treat a fungal infection or disease associated with one or more of the fungal pathogens described herein. By way of example, an effective amount of an isolated antiserum may be determined by measuring the amount or titer of antibodies directed against the desired immunogen present in the serum by methods known and practiced in the art. The range of typical dosages for passive immunotherapy (i.e., the administration of antiserum containing antibodies) includes about 0.3 mg to about 100 mg/kg of total body weight. Following passive immunotherapy, treatment efficacy is typically conducted, as individual patients respond differently to therapies. Adjustment of the dosage may be modified as needed. Treatment regimens can be determined by methods known and practiced by those having skill in the art.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

Fungal burden refers to the quantity of fungal organisms extant in a subject, or in a cell, tissue, organ, or sample, e.g., blood, plasma, serum, cerebrospinal fluid (CSF), bronchoalveolar lavage, pulmonary lavage, urine, sputum, and the like, of the subject. In an embodiment, the fungal organism is Aspergillus spp. or A. fumigatus. In an embodiment, the quantity of the fungal organism is determined by measuring colony forming units (CFU)/mL of the Aspergillus organism in a sample obtained from a subject. In embodiments, the obtained sample is lung tissue, bronchoalveolar lavage, or pulmonary lavage.

The terms “immunosuppressed” or “immunosuppression” refer to the partial or complete suppression (e.g., dampening) of the body's immune system and its ability to elicit an immune response to fight or ward off infections and other diseases. Immunosuppression in a patient may be congenital (e.g., primary immunodeficiency disease) or acquired (e.g., secondary immunodeficiency disease). Immunosuppression may result from certain diseases and medical conditions, e.g., inflammatory diseases, lymphoma, HIV-AIDS; cancer; or it may result from or be induced by treatment of an individual with drugs, e.g., anticancer drugs, medications, or other agents. Drug induced immunosuppression may be utilized in the treatment of diseases or conditions in a subject, for example, without limitation, inflammatory diseases, rheumatoid arthritis, psoriasis, eczema, systemic lupus erythematosus (“lupus”), anemia (e.g., autoimmune hemolytic anemias), inflammatory bowel disease (IBD), inflammatory bowel syndrome (IBS), Crohn's disease, colitis, cancer, leukemia and HIV-AIDS. By way of example, congenital immunosuppression may afflict an individual having an inherited disease that affects the immune system, e.g., ataxia-telangiectasia, complement deficiencies, congenital (e.g., X-linked) agammaglobulinemia, common variable immunodeficiency (CVID), congenital IgA deficiency, severe combined immunodeficiency (SCID), hypogammaglobulinemia, Job syndrome, DiGeorge syndrome, and the like. Nonlimiting examples of secondary immunodeficiency disorders include AIDS, cancers of the immune system, e.g., leukemia and B- and T-cell lymphomas and tumors, e.g., multiple myeloma, and immune complex diseases, e.g., viral hepatitis. In an embodiment, immunosuppression can be induced in a patient to aid in the survival of an organ or tissue following transplantation.

An immunosuppressive drug, agent, or medication refers to a drug, agent, or medication that inhibits or prevents activity of the immune system and components thereof. Classes of immunosuppressive drugs and the like include, without limitation, glucocorticoids, (corticosteroids, steroids), cytostatics (e.g., agents that inhibit cell division of immune cells, such as B- and T-cells), antibodies, and drugs that act on immunophilins. Nonlimiting examples of glucocorticoids include prednisone, dexamethasone and hydrocortisone. Nonlimiting examples of cytostatics include alkylating agents (e.g., nitrogen mustard, cyclophosphamide, nitrosoureas and platinum compounds); antimetabolites (e.g., folic acid analogs such as methotrexate, purine analogs such as azathioprine and mercaptopurine, pyrimidine analogs such as fluorouracil and inhibitors of protein synthesis); cytotoxic antibiotics (e.g., dactinomycin, anthracyclines, mitomycin C, bleomycin and mithramycin). Methotrexate, an antimetabolite of the antifolate type, is a chemotherapeutic agent and immune system suppressant and is used, either alone or in combination with other agents or drugs, to treat cancer, inflammatory diseases and autoimmune diseases. By way of nonlimiting example, methotrexate is administered for the treatment of bladder cancer, breast cancer, head and neck cancer, leukemia, lung cancer, lymphoma, osteosarcoma and trophoblastic neoplasms. In addition, methotrexate is used to treat inflammatory and autoimmune diseases such as, without limitation, eczema, rheumatoid arthritis, psoriasis, psoriatic arthritis, lupus, sarcoidosis, Behcet's disease, Crohn's disease and various forms of vasculitis. Antibodies, e.g., monoclonal, recombinant, polyclonal, single chain antibodies and the like, are used as a potent immunosuppressive therapy to prevent the acute rejection reactions, as well as a targeted treatment for lymphoproliferative or autoimmune disorders (e.g., anti-CD20 monoclonal antibodies). By way of further example, T-cell receptor directed antibodies, e.g., muromonab-CD3, are used as immunosuppressant agents to prevent T cell activation and proliferation by binding to the T cell receptor complex present on differentiated T cells. IL-2 receptor binding antibodies act as immunosuppressants by binding to the a chain of the IL-2 receptor, thereby preventing IL-2 cytokine-induced clonal expansion of activated lymphocytes and shortening their survival. Anti-IL-2 receptor antibodies may be used, for example, in the prophylaxis of the acute organ rejection after transplantation, e.g., bilateral kidney transplantation. Ciclosporin provides a nonlimiting example of a drug that acts on immunophilins. Ciclosporin binds to the cytosolic protein cyclophilin (an immunophilin) of immunocompetent lymphocytes, such as T cells. The complex of ciclosporin and cyclophilin inhibits the phosphatase calcineurin, which, under normal circumstances, induces the transcription of IL-2. The drug also inhibits lymphokine production and interleukin release, leading to a reduced function of effector T-cells. Other nonlimiting examples of immunosuppressive drugs or agents include tacrolimus (a calcineurin inhibitor); sirolimus (rapamycin), an inhibitor of T cell signal transduction and clonal proliferation; everolimus (an mTOR inhibitor); interferons (e.g., IFN-β; (suppresses Th1 cytokine production and monocyte activation); IFN-γ (triggers apoptosis of lymphocytes)); opioids; TNF-binding proteins (e.g., TNFα binding protein, which binds to and prevents TNFα from inducing IL-1 and IL-6 synthesis and from inducing adhesion of lymphocyte activating molecules); mycophenolate (e.g., mycophenolic acid, which inhibits inosine-5′-monophosphate dehydrogenase (IMPDH), a key enzyme in guanosine nucleotide synthesis); and small molecule agents (e.g., fingolimod, which modulates the activity of certain adhesion molecules such as α4/ß7 integrin, in lymphocytes, causing their accumulation in lymphatic tissue and decreased concentration in the circulation; or myriocin).

Immunosuppression may also be caused by or induced in an individual by treatment with anti-cancer, chemotherapeutic, anti-inflammatory, immunotherapy and/or immuno-oncology (TO) agents, drugs, therapies, or medicaments. Immuno-oncology (also called cancer immunotherapy) refers to the stimulation of the immune system and cells to treat cancer by enhancing or improving on the immune system's natural ability to respond to disease such as cancer and tumors. IO treatments exploit the presence of tumor antigens or molecules (such as proteins or other macromolecules (e.g. carbohydrates)) on the surface of cancer cells that can be bound and/or detected by antibody proteins of the immune system. Without wishing to be bound by theory, immunotherapeutic antibodies bind to tumor antigens, thereby marking and identifying the cancer cells as targets for inhibition or killing by cells of the immune system. Cancer-targeting immunotherapies have been developed to modify the immune system to recognize that a cancer or tumor is foreign to the body and must be attacked and eradicated. White blood cells express “immune checkpoint” molecules that alert cells to either “engage and fight” or “ignore and rest” when a foreign agent is recognized in the body. Under normal circumstances, immune checkpoint molecules prevent the immune system from attacking normal cells. IO drugs called checkpoint inhibitors block these molecules, allowing the immune cells to start attacking cancer cells. By way of nonlimiting example, IO drugs or agents include immune checkpoint inhibitors, such as antibodies (e.g., monoclonal antibodies), antibodies directed against CTLA-4 (ipilimumab), or binding agents (e.g., antibodies) that inhibit or disrupt the interaction of PD-1 with PD-L1. Examples of anti-PD-1/PD-L1 agents include, without limitation, pembrolizumab (Keytruda), nivolumab (Opdivo), durvalumab (Imfinzi), atezolizumab (Tecentriq), avelumab (Bavencio), cemiplimab, sintilimab, toripalimab and camrelizumab, ipilimumab (Yervoy), used alone or in combination with other agents (biologics), small molecule compounds, or drugs (Yu, J. X. et al., 2019, Nature Reviews, Drug Discovery; doi: 10.1038/41573-019-00182-w). As another example, alemtuzumab, which is used to treat chronic lymphocytic leukemia (CLL) and multiple sclerosis, is a monoclonal antibody that binds to CD52 on the surface of mature lymphocytes, but not stem cells. Following binding by alemtuzumab, the CD52-bearing lymphocytes are targeted for destruction. In addition, chimeric antigen receptor (CAR) T cells are used to treat patients with cancers and/or tumors, such as hematologic cancers and malignancies, by means of an adoptive cell transfer approach.

By “immunocompromised” is meant having a weakened or impaired immune system, for example, as a result of drugs, medications or other agents, or as a result of illness, disease or pathology. An individual who is immunocompromised has a reduced ability to fight or ward off infection and disease and to elicit or mount an immune response against infection and disease or disease-causing agents.

The terms “isolated,” “purified,” or “biologically pure” refer to material that is free to varying degrees from components which normally accompany it as found in its native state or environment. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” or “isolated peptide” is meant a polypeptide or peptide that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, collecting, isolating, or otherwise acquiring the agent.

By “reduces” or “diminishes’ is meant a negative alteration of at least 10%, 25%, 50%, 75%, or 100%.

By “reference” is meant a standard or control condition. A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody or antigen binding fragment thereof that recognizes and binds a polypeptide or peptide, but which does not substantially recognize and bind other molecules in a sample, for example, a biological sample, which naturally includes a polypeptide of the invention. Cross-reactive binding includes specific binding (e.g., by an antibody or an antigen binding fragment thereof) to an original polypeptide or peptide antigen/immunogen as well as binding to a polypeptide or peptide other than the original antigen/immunogen.

Nucleic acid molecules useful in generating a recombinant immunogen or a vaccine include any nucleic acid molecule that encodes a polypeptide or a peptide fragment thereof, such as an A. fumigatus Kexin polypeptide or peptide described herein. Such nucleic acid molecules need not be 100% identical with an endogenous nucleic acid sequence, but will typically exhibit substantial identity to an endogenous sequence. Polynucleotides having “substantial identity” to an endogenous sequence are typically capable of hybridizing with at least one strand of a double-stranded nucleic acid molecule. Nucleic acid molecules may include any nucleic acid molecule that encodes a polypeptide or a peptide fragment thereof. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a non-human primate, or a murine, bovine, equine, canine, ovine, or feline mammal. In an embodiment, the subject is a veterinary subject. In an embodiment, the subject is a human. In an embodiment, a subject is a human individual or patient who is undergoing treatment for or is at risk of Aspergillus infection or disease caused by an Aspergillus fungal pathogen, such as aspergillosis. In an embodiment, the Aspergillus fungal pathogen is A. fumigatus. In an embodiment as subject is a human patient who is susceptible to or at risk of infection (e.g., opportunistic infection) or disease caused by Aspergillus, e.g., A. fumigatus. In an embodiment, the subject is a mammalian (e.g., a human; a non-human primate) donor subject from whom antiserum containing anti Aspergillus KEX peptide antibodies is obtained or isolated. In an embodiment, the subject is a mammalian (e.g., a human; a non-human primate) recipient subject who receives an isolated antiserum containing anti-Aspergillus Kexin peptide antibodies and who, in turn, acquires protective immunity (and treatment) against aspergillosis disease. In an embodiment, the anti-Aspergillus KEX peptide antibodies are anti-A. fumigatus KEX peptide antibodies. In an embodiment, the A. fumigatus KEX peptide against which antibodies are generated is the 90-mer AF.KEX1 peptide (SEQ ID NO: 2). In an embodiment, the A. fumigatus KEX peptide against which antibodies are generated is the 88-mer A. fumigatus KEX peptide (SEQ ID NO: 3).

By “opportunistic infection” is meant an infection caused by pathogens such as fungal pathogens, bacteria, viruses, protozoa, or parasites that take advantage of an opportunity to infect a subject (host) that is not normally available, for example, a host having a weakened immune system, an immunocompromised host, a host with altered microbiota or microflora, or a host having protective integumentary barriers that have been damaged or breached. In an embodiment, an opportunistic infection is caused by one or more fungal pathogens as described herein.

As used herein, “PS-15” refers to a dihydrofolate reductase inhibitor having the following structure:

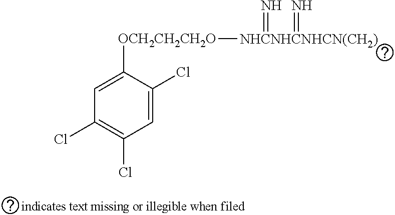

“QS-21” refers to an adjuvant having the following structure:

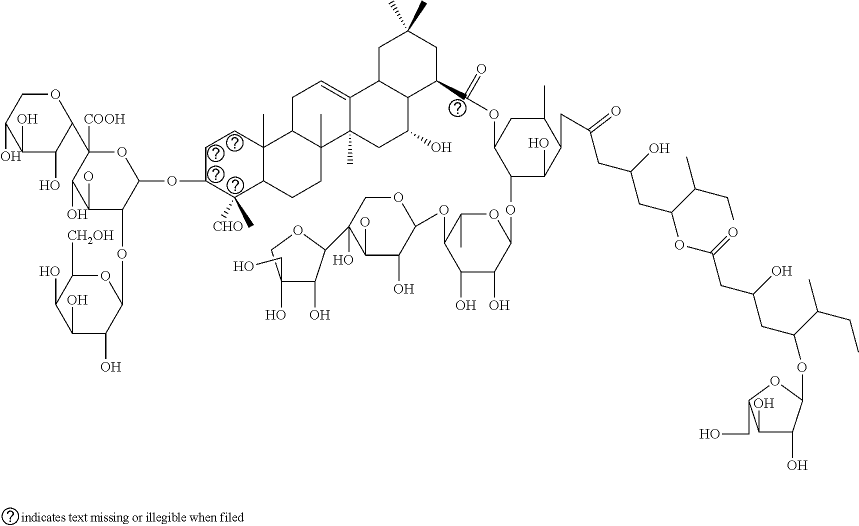

Soltysik et al., Structure/Function Studies of QS-21 Adjuvant: Assessment of Triterpene Aldehyde and Glucuronic Acid Roles in Adjuvant Function, Vaccine, 13(15): 1403-1410 (1995). QS-21, a purified plant extract that enhances the ability of the immune system to respond to immunogens and vaccine antigen, is derived from the Chilean soap bark tree (Quillaja saponaria). The extract contains water soluble triterpene glycoside compounds (saponins) and is an immunologic adjuvant.

Ranges provided herein are understood to be shorthand for all the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing, abating, diminishing, or ameliorating a disease, disorder and/or symptoms associated therewith. It will be appreciated that, although not precluded, treating a disease, disorder and/or symptoms associated therewith does not require that the disease, disorder, condition or symptoms associated therewith be completely eliminated.

As used herein, a therapeutic that “prevents” a disorder or condition refers to a compound or material that, in a statistical sample, reduces the occurrence of the disorder or condition in the treated sample relative to an untreated control sample, or delays the onset or reduces the severity of one or more symptoms of the disorder or condition relative to an untreated control sample. In an embodiment, a preventive therapeutic is an antibody or an antigen binding fragment thereof. In an embodiment, a preventive therapeutic is an isolated antiserum containing anti-Aspergillus KEX peptide antibodies or antigen binding fragments thereof as described herein. In an embodiment, the isolated antiserum contains anti-Aspergillus fumigatus KEX peptide antibodies or antigen binding fragments thereof.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

### BRIEF DESCRIPTION OF THE DRAWINGS

Exemplary embodiments of the present invention are provided below as drawings and figures related to the description of the invention in its various and nonlimiting aspects.

FIG. 1 provides an alignment showing the 90 amino acid Kexin peptide (AF.KEX1, SEQ ID NO: 2), derived from the Aspergillus fumigatus Kexin polypeptide, and the 88 amino acid A. fumigatus Kexin peptide (A. fumigatus KEX peptide, SEQ ID NO: 3). By way of example, an A. fumigatus KEX peptide of 90 amino acids containing an N-terminal TEV cleavage side and maltose binding protein (MBP) tag (Aspergillus KEX-MBP) was recombinantly expressed and affinity purified. (See, e.g., Example 2). The purified recombinant protein was then incubated with TEV protease to cleave the N-terminal MBP affinity tag (Aspergillus KEX-MBP+TEV). This reagent was suitable for use in immunoblotting studies following resolution of recombinant proteins by 15% SDS-PAGE.

FIGS. 2A and 2B present immunohistochemical staining and radiographic images reflecting infection of lung tissue by Aspergillus fumigatus, which is an opportunistic fungal pathogen that can cause invasive pulmonary disease in immunocompromised individuals. FIG. 2A shows hematoxylin and eosin (H&E) staining caused by Aspergillus hyphae invading through the bronchial wall. FIG. 2B shows a radiograph of the chest cavity of an immunocompromised subject infected with Aspergillus, in which fibrosis and a fungal ball can be observed.

FIGS. 3A and 3B illustrate Western blots showing Aspergillus fumigatus Kexin peptide (AF.KEX1) specific humoral responses following immunization of mice with AF.KEX1 peptide immunogen and challenge with A. fumigatus organisms. Shown are developed Western blots in which recombinant AF.KEX1 protein (demarcated with asterisks) was contacted with the plasma of a single mouse prior to (FIG. 3A, naïve) and following (FIG. 3B, immunized) challenge with A. fumigatus Af293 pathogenic fungus.

FIGS. 4A-4E illustrate a study design for immunizing (vaccinating) mice, graphs and a Western blot depicting anti-A. fumigatus Kexin peptide antibody titer versus days post immunization of animals in the study. FIG. 4A presents the immunization/vaccination study design utilizing the AF.KEX1 peptide immunogen. FIG. 4B depicts a graph showing AF.KEX1-specific immunoglobulin G (IgG) titer in plasma of animals, as determined by enzyme-linked immunosorbent assay (ELISA) of a single immunization/vaccination of the animals with AF.KEX1+TITERMAX1 and PBS+TITERMAX. FIG. 4C depicts a graph of the results following two immunizations (vaccinations) of animals with AF.KEX1 immunogen+TITERMAX. The days on which animals were administered (immunized or vaccinated with) the AF.KEX1 immunogen are indicated with downward arrows. Mann-Whitney rank tests were performed to compare data baseline to data. All post-vaccination timepoints for Groups 1 and 3 were p<0.0001, compared with baseline. FIG. 4D shows Western blots of recombinant AF.KEX1 peptide (lane 2 of each blot) and KEX1 (lane 3 of each blot) developed using plasma obtained from mice 28 days post immunization with AF.KEX1 peptide immunogen+TITERMAX (“AF.KEX1-Vaccinated,” left blot) or with PBS+TITERMAX (“Sham Vaccinated,” right blot). The study demonstrated AF.KEX1-specific humoral responses in animals following immunization of mice with AF.KEX1 immunogen and subsequent challenge of the animals with A. fumigatus organisms. FIG. 4E (top) presents a bar graph showing a comparison of anti-AF.KEX1 peptide antibody titers in animals that had received one immunization of AF.KEX1 peptide immunogen and animals that had received 2 immunizations of AF.KEX1 peptide immunogen (an initial immunization and a boost) at 28 days (See, e.g., Example 6). FIG. 4E (bottom) presents a bar graph showing antibody titers in sham-immunized control animals (animals that had received no AF.KEX1 peptide immunogen and animals that had received only PBS+TITERMAX).

FIGS. 5A and 5B illustrate a study design for immunizing (vaccinating) mice and challenging the immunized animals with A. fumigatus organisms thereafter, and a graph showing survival curves of animals after challenge. FIG. 5A presents the immunization/challenge study design. FIG. 5B depicts survival curves (Kaplan-Meier curves) of A. fumigatus-challenged animals that had been immunized with the A. fumigatus Kexin peptide (AF.KEX1), e.g., the 90-mer A. fumigatus Kexin peptide, compared with animals that did not receive the AF.KEX1 immunogen (SHAM (mock)-treated control animals) prior to challenge. The results demonstrate that the AF.KEX1-immunized animals were significantly protected from developing Aspergillosis disease, compared with mock-immunized controls (p=0.0487, by Mantel-Cox test).

FIGS. 6A-6D present graphs and stained tissue images related to lung fungal burden following challenge of mice immunized with A. fumigatus KEX1 peptide immunogen. FIG. 6A shows a plot reflecting the quantification of the fungal burden in the lungs of animals immunized with the AF.KEX1 peptide or Sham (PCS) control. Results are based on the use of Grocott's methenamine silver (GMS) staining. FIG. 6B provides a plot showing the results of qPCR of fungal DNA present in lungs of AF.KEX1 immunized and mock-treated (Sham) animals. FIG. 6C presents images of lung tissue following GMS staining of the lungs of mice post-immunization (vaccination) with AF.KEX1 immunogen and Af293 A. fumigatus challenge. (20× magnification with 4× insert). Mann-Whitney rank tests were performed to compare cohorts. **p<0.01, *p<0.05. FIG. 6D presents images of lung tissue following GMS staining of the lungs of mock-treated (SHAM; no AF.KEX1 immunization) and Af293 A. fumigatus challenge (20× magnification with 4× insert). Mann-Whitney rank tests were performed to compare cohorts. **p<0.01,*p<0.05.

FIG. 7 depicts a plot showing the correlation of anti-AF.KEX1 antibody titers with fungal burden in lungs of study animals. Spearman correlation indicated a significant negative correlation between antibody titer and fungal burden (r=−0.8284, p<0.001). The results show that the antibody titer generated in a mammalian subject following immunization with AF.KEX1 peptide was inversely correlated with the lung fungal burden.

FIG. 8 presents a graph showing survival curves (Kaplan-Meier curves) of A. fumigatus-challenged mice that had been immunized with the A. fumigatus KEX1 peptide (AF.KEX1) compared with mice that did not receive the AF.KEX1 immunogen (SHAM-treated control animals) prior to immunosuppression with FK506 and hydrocortisone and A. fumigatus challenge. The results demonstrate that the AF.KEX1-immunized animals were significantly protected from developing aspergillosis disease compared with sham-immunized controls (p=0.0183, by Mantel-Cox test).

### DETAILED DESCRIPTION OF THE EMBODIMENTS

Aspergillus, a common mold, causes aspergillosis, allergic reactions, lung infections and other health problems. Nearly 40 of the approximately 180 species of Aspergillus fungal pathogens cause infections in humans. Examples of species of Aspergillus fungal organisms include, without limitation, Aspergillus fumigatus (A. fumigatus), Aspergillus flavus (A. flavus), Aspergillus terreus (A. terreus), Aspergillus nidulans (A. nidulans), Aspergillus versicolor (A. versicolor) and Aspergillus niger (A. niger).

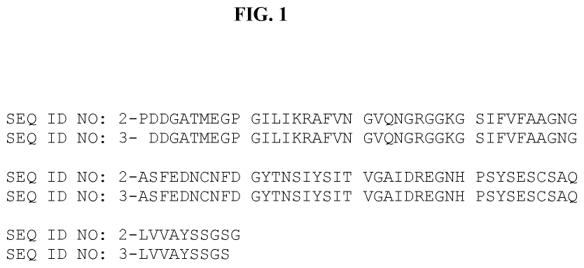

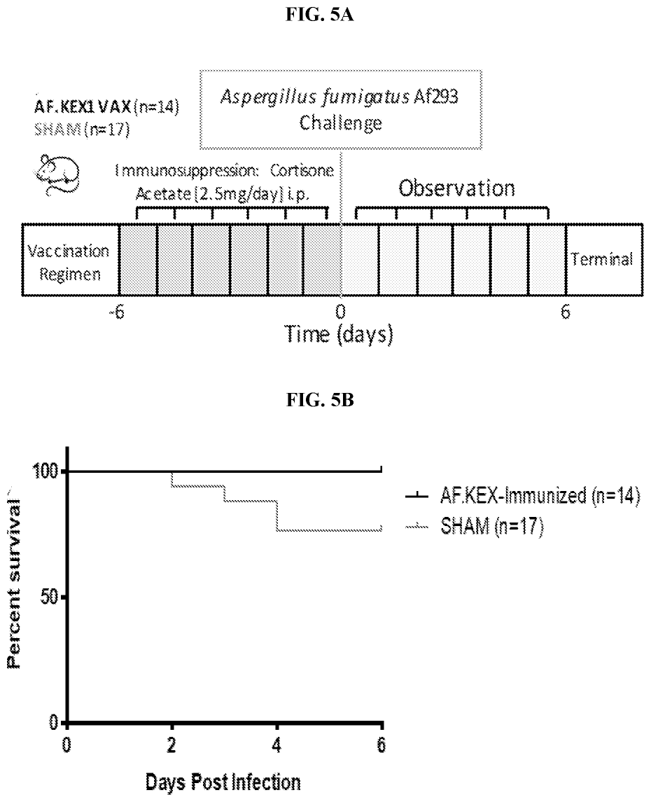

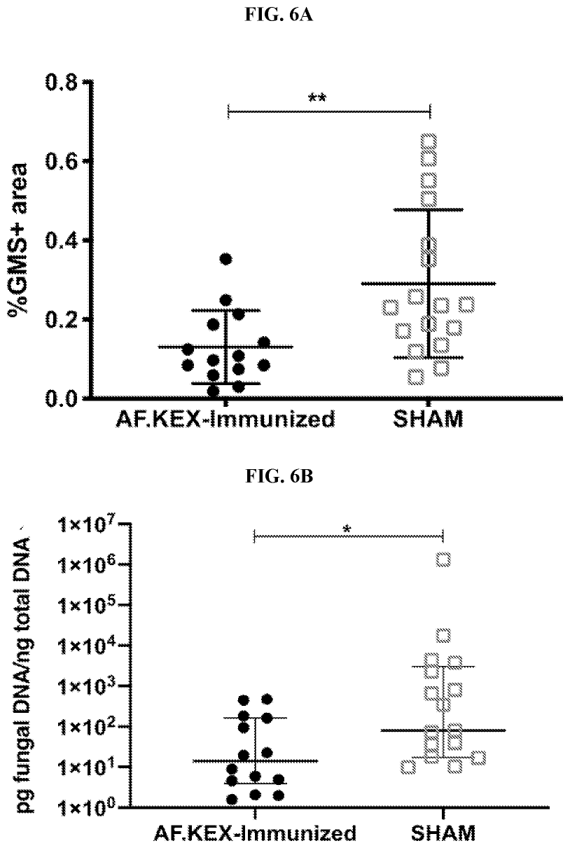

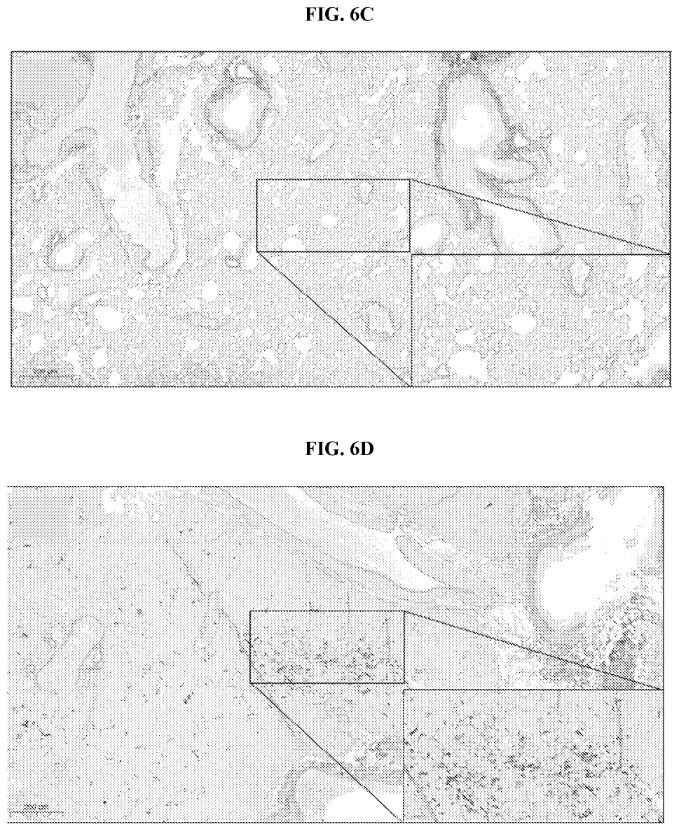

The methods described herein involve the use of a Kexin peptide derived from the Kexin (KEX) protein of an Aspergillus fungal organism. The Aspergillus Kexin peptide used as an immunogen was demonstrated to provide therapeutic and/or protective treatment against aspergillosis and/or its symptoms following administration, e.g., immunization or inoculation, to subjects in need thereof (FIGS. 5B and 6A-6D). In an embodiment, the Kexin peptide derived from A. fumigatus is a 90 amino acid sequence, called AF.KEX1 herein, comprising or consisting of SEQ ID NO: 2 (FIG. 1). In an embodiment, the A. fumigatus KEX peptide is an 88 amino acid sequence comprising or consisting of SEQ ID NO: 3 (FIG. 1). In an embodiment, the Aspergillus Kexin peptide is a recombinant protein. In an embodiment, a polynucleotide encoding the 90- or 88-mer A. fumigatus KEX peptide is harbored in an expression vector, which expresses the peptide. In an embodiment, the A. fumigatus KEX peptide (e.g., AF.KEX1) peptide is suitable for use as a vaccine, e.g., a protective immunogen, to treat or protect against aspergillosis in subjects in need thereof, in particular, immunocompromised or immunosuppressed patients. In an embodiment, the A. fumigatus KEX peptide (e.g., AF.KEX1) peptide is administered in conjunction with an adjuvant. In an embodiment, the adjuvant is a TITERMAX adjuvant. In an embodiment, the Aspergillus Kexin peptide is administered to a human subject or patient.

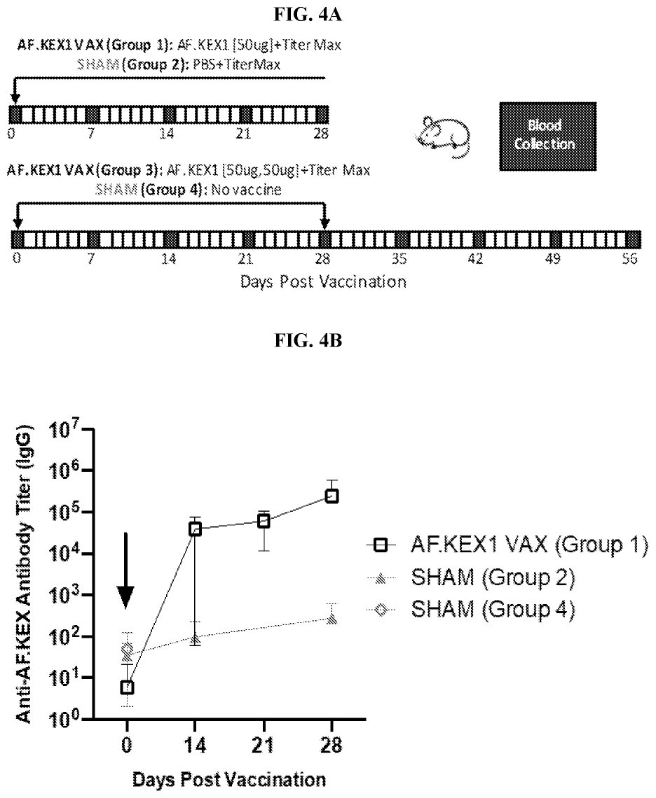

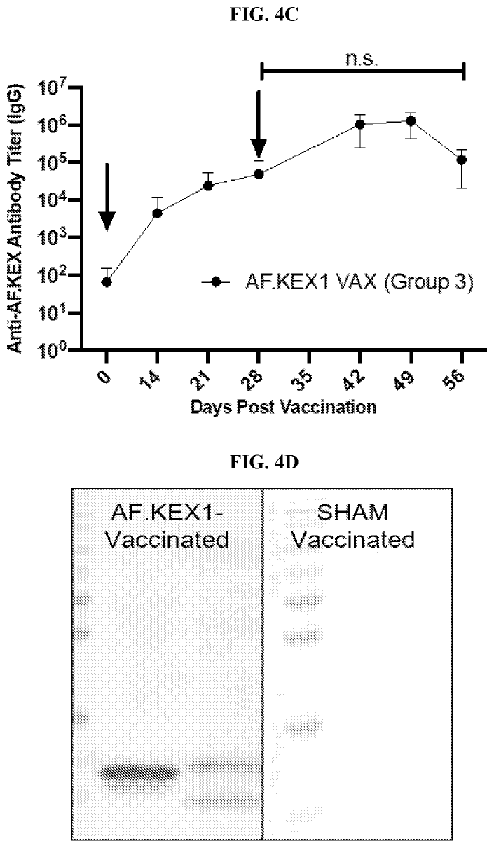

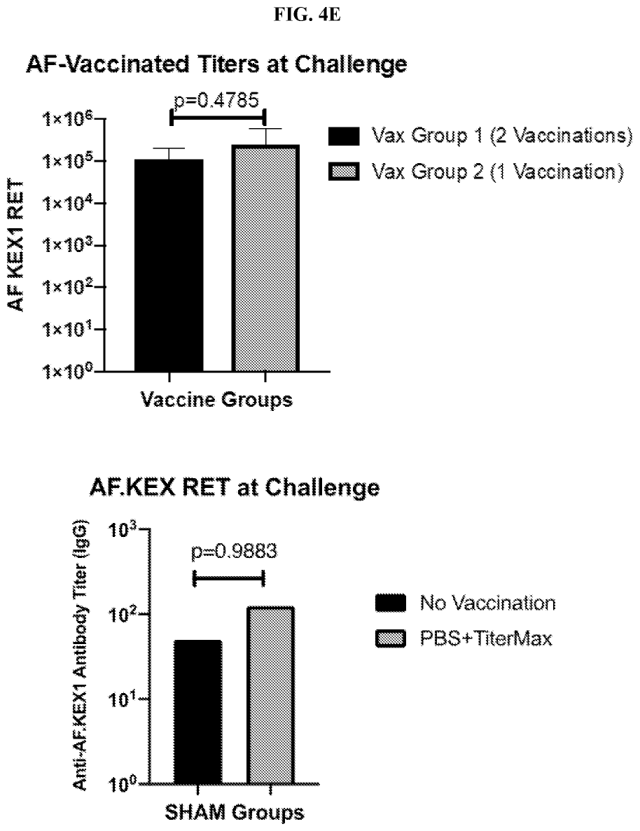

In an aspect, the Aspergillus Kexin peptide (e.g., AF.KEX1), as immunogen, administered to a healthy mammalian subject generated a robust immune response, e.g., a robust antibody response, in the subject (FIGS. 4B and 4C). In an aspect, the Aspergillus Kexin peptide (e.g., AF.KEX1 peptide) administered to a mammalian subject significantly reduced A. fumigatus-induced mortality and lung fungal burden in the subject infected with A. fumigatus pathogenic fungi, e.g., in a murine model of invasive pulmonary aspergillosis (IPA). Thus, vaccination (i.e., immunization or inoculation) of a mammalian subject with the AF.KEX1 peptide as immunogen generated a potent antibody response that significantly reduced both mortality and lung fungal burden in the A. fumigatus-infected subject afflicted with IPA. In another aspect, the Aspergillus Kexin peptide (e.g., AF.KEX1 peptide) administered to an immunosuppressed mammalian subject generated a protective immune response following cortisone acetate-induced immunosuppression and A. fumigatus challenge, e.g., in an immunosuppressed murine model. In an embodiment, an adjuvant, e.g., TITERMAX, was also administered to the subject, e.g., to enhance the generation of an immune response. Moreover, the lung fungal burden in the subject was found to be inversely correlated with the peak antibody titer achieved following immunization (vaccination) of the mammalian subject with the Aspergillus Kexin peptide. In an embodiment, the Aspergillus Kexin peptide is administered to a human subject or patient.

The practice of the described methods supports the advantages and benefits of the Aspergillus Kexin peptide (e.g., AF.KEX1 peptide) as a biologic therapeutic to treat and prevent aspergillosis, such as IPA, in mammalian subjects in need thereof. In a particular embodiment, the mammalian subjects are immunosuppressed or have undergone drug-induced immunosuppression. The results further support the advantages and benefits of the Aspergillus Kexin peptide (e.g., AF.KEX1 peptide) vaccination or immunization as an alternative treatment for the prevention of aspergillosis, such as IPA, in immunosuppressed mammalian subjects, e.g., drug-induced immunosuppressed subjects. In an embodiment, the subject is a human patient.

Methods are also provided for treating aspergillosis disease and/or the symptoms thereof in a mammalian subject who is infected with an Aspergillus fungal pathogen, in which the method involves administering to the subject an effective amount of an Aspergillus Kexin peptide (e.g., AF.KEX1 peptide) to treat aspergillosis disease and/or the symptoms thereof in the subject. In an embodiment, the aspergillosis disease is one or more of allergic bronchopulmonary aspergillosis (ABPA), allergic Aspergillus sinusitis, aspergilloma, chronic pulmonary aspergillosis, invasive pulmonary aspergillosis (IPA), or cutaneous aspergillosis. In a particular embodiment, the aspergillosis disease is IPA. In embodiments, passive immunization against Aspergillus (e.g., A. fumigatus) infection and/or aspergillosis disease and its symptoms may be provided to a subject to whom an isolated antiserum comprising antibodies generated against Aspergillus Kexin peptide as described herein is administered.

The methods embrace the use of an immunogenic composition comprising a Kexin peptide derived from Aspergillus spp., e.g., Aspergillus fumigatus, that elicits a potent immune response in a subject following administration of the composition and the production of antiserum in the subject that contains antibodies or antigen binding fragments thereof that specifically react with the immunizing Aspergillus Kexin peptide as immunogen. In an embodiment, the Aspergillus Kexin peptide comprises or consists of SEQ ID NO: 2. In an embodiment, the Aspergillus Kexin peptide comprises or consists of SEQ ID NO: 3.

The methods and compositions described herein provide economic, medical and practical benefits in the treatment and prevention of Aspergillus fungal infection and aspergillosis disease, e.g., invasive pulmonary aspergillosis (IPA) or pulmonary disease caused by Aspergillus colonization in the lung.

### Types of Aspergillosis

Aspergillosis is a disease caused by the fungal pathogen, Aspergillus, a common mold that exists both indoors and outdoors. Aspergillosis may be contracted by inhalation of microscopic, air-borne Aspergillus fungal spores from the environment into the lungs or sinuses; however, aspergillosis is not directly communicable between individuals or between individuals and non-human animals from the lungs. Hospital-acquired (nosocomial) Aspergillus infections may be sporadic, or they may be associated with dust exposure during building renovation or construction. Occasional outbreaks of cutaneous infection by Aspergillus have been traced to contaminated biomedical devices. The incubation period for aspergillosis is unclear and may vary depending on the dose of Aspergillus and the host immune response.

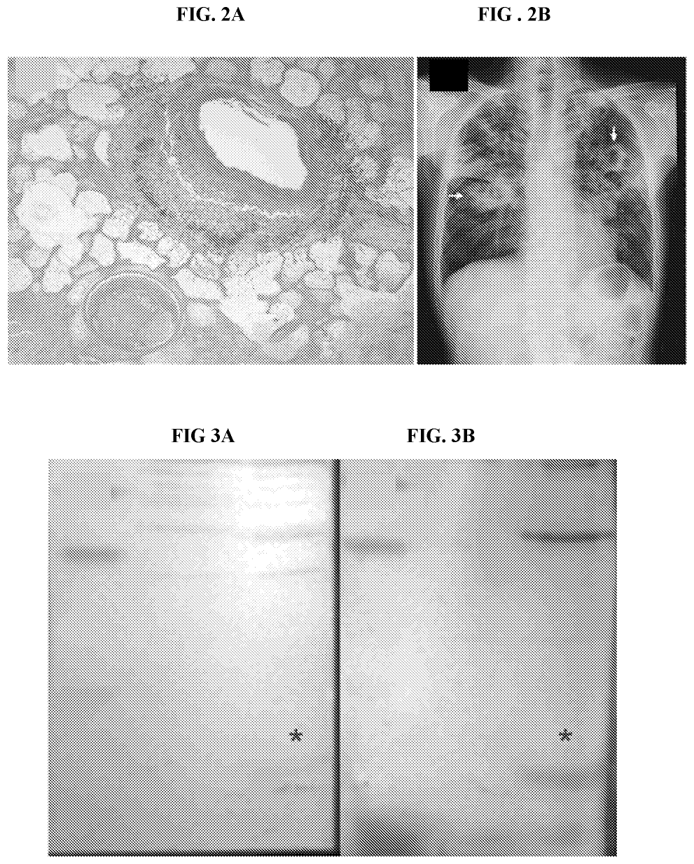

Individuals who are immunocompromised by having weakened immune systems or lung diseases are typically at a higher risk of developing health problems and diseases associated with the presence of Aspergillus in the body, with infection of cells and tissues by Aspergillus (FIG. 2A) and with the inhalation of Aspergillus spores (conidia), compared with individuals with healthy immune systems.

Individuals (e.g., patients) can present with different types of aspergillosis, which can range from mild to extremely serious. By way of example, different types of aspergillosis diseases include allergic bronchopulmonary aspergillosis (ABPA) in which the Aspergillus fungus causes inflammation in the lungs and allergy symptoms such as coughing and wheezing; allergic Aspergillus sinusitis, in which Aspergillus causes inflammation in the sinuses and symptoms of a sinus infection (drainage, stuffiness, headache); aspergilloma, also called a “fungus ball,” which constitutes a ball of Aspergillus fungal organisms that grows in the lungs or sinuses (FIG. 2B), but usually does not spread to other parts of the body; and chronic pulmonary aspergillosis, which is a long-term condition in which Aspergillus can cause cavities in the lungs and in which one or more fungal balls (aspergillomas) may also be present in the lungs. Chronic pulmonary aspergillosis may last for days, weeks, or months, e.g., 3 months or longer.

Another type of aspergillosis is invasive pulmonary aspergillosis (IPA), a serious infection that usually affects individuals who have weakened immune systems, for example, those who have had an organ transplant or a stem cell transplant. IPA most commonly affects the lungs, although it can also spread to other parts of the body. With regard to invasive aspergillosis, over 10 million patients in the United States, Europe and Japan are at risk of developing or having invasive aspergillosis (IPA) each year as a result of corticosteroid or other therapies. Despite existing treatments as of 2017, the mortality rate of patients with IPA is over 50%. Annually, the patients who develop IPA worldwide (e.g., in the United States, Europe, China and Japan) are frequently afflicted with diseases such as acute leukemia, stem cell and other transplants, chronic obstructive pulmonary disease (COPD), lung cancer, liver failure, lymphoma, chronic leukemia, immunological disorders, and drug treatments. Yet another type of aspergillosis includes cutaneous (skin) aspergillosis, a disease caused by the entry of Aspergillus fungus into the body through a break in the skin (e.g., following surgery; through a wound, or a burn wound), causing infection. Individuals who are immunocompromised and weakened immune systems are particularly vulnerable and susceptible to contracting cutaneous aspergillosis. Moreover, cutaneous aspergillosis can occur if invasive pulmonary aspergillosis spreads to the skin from somewhere else in the body, such as the lungs.

The different types of aspergillosis disease can cause different symptoms in afflicted individuals. By way of nonlimiting example, the symptoms of allergic bronchopulmonary aspergillosis (ABPA) are similar to asthma symptoms and may include wheezing, shortness of breath, coughing and, in some cases, fever. The symptoms of allergic Aspergillus sinusitis may include stuffiness, runny nose, headache and a reduced ability to smell. The symptoms of aspergilloma (fungus ball) may include a cough, coughing up blood and shortness of breath. The symptoms of chronic pulmonary aspergillosis may include weight loss, cough, coughing up blood, fatigue and shortness of breath. Invasive pulmonary aspergillosis frequently occurs in individuals or patients who are previously ill from other medical conditions; therefore, it may be difficult to discern which symptoms are related to an Aspergillus infection. Notwithstanding, invasive pulmonary aspergillosis does have diagnosable symptoms, which include, without limitation, fever, chest pain, cough, coughing up blood and shortness of breath. Other symptoms can develop in the patient if the infection spreads from the lungs to other parts of the body.

### Diagnosis of Aspergillosis

When diagnosing aspergillosis, a healthcare provider typically considers an individual's medical history, risk factors, symptoms, physical examination, and laboratory test results. In some cases, imaging tests, such as a chest x-ray or a CT scan of a patient's lungs or other parts of the body may be required, depending on the location of the suspected infection. If a healthcare provider suspects an Aspergillus infection in the lungs of a patient, a fluid sample from the respiratory system is collected for laboratory analysis and confirmed diagnosis. Healthcare providers may also perform a tissue biopsy, in which a small sample of affected tissue is analyzed in a laboratory under a microscope or in a fungal culture for evidence of the presence of Aspergillus organisms. A blood test can assist in the early diagnosis of invasive pulmonary aspergillosis (IPA) in individuals who have severely weakened immune systems.

A definitive diagnosis of aspergillosis typically requires a positive culture from a normally sterile site and histopathological evidence of infection. Other diagnostic tools include radiology, galactomannan antigen detection, Beta-D-glucan detection, and polymerase chain reaction (PCR). Microscopy is a nonlimiting example of a method and tool that is employed in the diagnosis of aspergillosis. Microscopy allows for the evaluation of respiratory specimens after the application of special stains for visualization of Aspergillus structures that appear as septated hyphae with acute angle branching. Microscopic identification may be used concurrently with other diagnostic methods and tools to increase sensitivity and to reduce the likelihood of false positives resulting from similarities in microscopic appearance to other types of filamentous fungi. Histopathology is employed to document the source(s) of invasive disease. In a manner similar to that of microscopy, Aspergillus appears as septated hyphae with acute angle branching and can be mistaken histopathologically for other filamentous molds. Organism culture may be carried out on a variety of sterile specimens or biological samples obtained from a subject. Aspergillus spp. present as rapidly growing molds that are visible 1-3 days after incubation. Culture allows for the microscopic identification of Aspergillus at the species level; however, because culturing methods can be relatively insensitive, patients with invasive pulmonary aspergillosis may have negative cultures. The Galactomannan antigen test is used to detect a polysaccharide that constitutes part of the cell wall of Aspergillus spp. and other fungi. The Platelia (Bio-Rad Laboratories) assay is approved by the US Food and Drug Administration (FDA) for assay of serum and bronchoalveolar lavage fluid. False positive test results have been reported in association with the administration of certain antibiotics, and cross reactivity with other infection-causing fungi may occur, such as Fusarium spp. or Histoplasma capsulatum. The beta-d-glucan assay is also used to detect a component in the cell wall of Aspergillus spp, as well as other fungi. The FUNGITELL® assay has been approved by the FDA for diagnosis of invasive fungal infections, including those due to Aspergillus fungal organisms. Similar to galactomannan testing, the specificity of this assay is reduced in a variety of clinical settings, including exposure to certain antibiotics, hemodialysis, and co-infection with certain bacteria. Polymerase Chain Reaction (PCR) is an efficient method for the detection of Aspergillus spp. from clinical specimens, including tissue and bronchoalveolar lavage fluid.

### Therapeutic and Protective Methods

The methods, biologic products and compositions provided herein can be used to treat or protect a mammalian subject in need thereof against Aspergillus infection and/or associated disease, e.g., aspergillosis, caused by the Aspergillus fungal pathogen, in particular, Aspergillus fumigatus. In embodiments, the methods, products and compositions described herein can provide immune protection in a subject against infection and disease caused by Aspergillus, e.g., aspergillosis, in particular, invasive pulmonary aspergillosis (IPA). Subjects immunized with (administered) an effective amount of an Aspergillus Kexin peptide, in particular, the AF.KEX1 peptide, generate an immune response in the form of antibodies specific for the Aspergillus Kexin peptide. Such antibodies are of high titer and comprise antisera which serve to treat and protect the subject against aspergillosis, e.g., IPA, or the development of aspergillosis, e.g., IPA; and/or reduce the severity of aspergillosis disease and/or its symptoms, and/or protect against the development of or reduce the severity of colonization of the lung by Aspergillus organisms and/or reduce Aspergillus fungal burden in the lungs of subjects immunized with (administered) the Aspergillus Kexin peptide. The methods, products and compositions described herein can protect a recipient subject, e.g., immunize or vaccinate, against development of aspergillosis disease caused by Aspergillus fungal organisms in the body. In an embodiment, the Aspergillus fungal pathogen is A. fumigatus. In an embodiment, the A. fumigatus Kexin peptide, AF.KEX1, comprises or consists of the amino acid sequence of SEQ ID NO: 2. In an embodiment, the A. fumigatus Kexin peptide comprises or consists of the amino acid sequence of SEQ ID NO: 3. In an embodiment, the aspergillosis disease associated with and/or caused by Aspergillus in the body includes the different types of aspergillosis described herein, and, in particular, IPA. In an embodiment, the subject is a human patient. In an embodiment, the subject is immunosuppressed or immunocompromised.

In an embodiment, antiserum isolated from an individual who had been immunized with an Aspergillus KEX1 peptide (e.g., AF.KEX1), in which the antiserum contains antibodies generated against the Aspergillus KEX peptide can be administered therapeutically and/or prophylactically to another individual in need thereof to provide immunity against aspergillosis disease, such as IPA. The methods include administering an immunologically effective amount of the isolated antiserum, or immune serum or immune plasma, described herein to an individual, alone, or in a physiologically acceptable carrier, excipient, or diluent. In an embodiment, the isolated antiserum is in a pharmaceutically acceptable composition.

In embodiments, the methods include the step of administering to a mammalian subject an effective amount of Aspergillus KEX peptide (e.g., AF.KEX1) to elicit an immune response in the subject in the form of specific anti Aspergillus KEX peptide antibodies, which treat Aspergillus infection and aspergillosis disease and/or its symptoms, and/or which protect against the development of aspergillosis in the subject. In an embodiment, an isolated antiserum containing antibodies generated against Aspergillus KEX peptide (e.g., AF.KEX1) is administered to a subject in need thereof in an amount effective to treat Aspergillus infection and/or aspergillosis disease and/or the symptoms thereof. In an embodiment, the Aspergillus KEX peptide (e.g., AF.KEX1) is in a pharmaceutically acceptable composition. In an embodiment, the isolated antiserum is in a pharmaceutically acceptable composition. In an embodiment, the subject is a human patient in need of treatment. In an embodiment, the human patient is immunosuppressed or immunocompromised.

Treatment will be suitably administered to subjects, particularly humans, suffering from, having, susceptible to, or at risk for infection by, an Aspergillus fungal organism, in particular, Aspergillus fumigatus, or aspergillosis disease. Determination of those subjects who are “at risk” can be made by any objective or subjective determination, e.g., by a diagnostic test or opinion of a subject or health care provider (e.g., genetic test, enzyme test or assay, or protein marker (such as levels of anti-Aspergillus KEX peptide antibodies, e.g., in serum), family history, and the like. Identifying a subject in need of treatment can involve the judgment of the recipient subject or a health care or medical professional and can be subjective (e.g., opinion) or objective (e.g., measurable by a test or diagnostic method).

In an embodiment, the methods involve treating or protecting against Aspergillus infection, aspergillosis disease and/or the symptoms thereof in a patient who is immunosuppressed or immunocompromised and/or who is receiving or has received immune suppressive drugs or medication and who, as a result of drug-induced immune system suppression, is susceptible to or may become susceptible to (or at risk of) Aspergillus infection or aspergillosis disease, either in or outside of a nosocomial environment. The type or cause of immunosuppression in a patient is not intended to be limiting. In general, an immunosuppressed or immunocompromised patient is more vulnerable, susceptible to, or at risk of Aspergillus infection, e.g., A. fumigatus infection, and/or having or developing aspergillosis disease. By way of nonlimiting example, immunosuppression in a patient may be congenital (e.g., resulting from primary immunodeficiency disease) or acquired (e.g., resulting from secondary immunodeficiency disease). Immunosuppression in a patient may result from certain diseases and medical conditions, e.g., inflammatory diseases, lymphoma, HIV-AIDS; cancer; or it may result from or be induced by treatment of an individual with drugs or medicaments (immunosuppressive drugs or medicaments), e.g., anticancer drugs, anti-inflammatory drugs, medications, or other agents or compounds, as described supra. In an embodiment, an immunosuppressed patient has a disease, illness, or condition that results in immunosuppression. In an embodiment, an immunosuppressed patient is being treated or receiving therapy for a disease, illness, or condition that results in immunosuppression. In an embodiment, an immunosuppressed patient is receiving drugs, e.g., anticancer or anti-inflammation drugs, medicaments, agents or compounds. In an embodiment, a patient is preparing to undergo a transplant (a pre-transplant patient) or may have received a transplant (a post-transplant patient) and is administered one or more immunosuppressive drugs or medications (anti-rejection medications) and/or is otherwise treated with drugs to reduce the likelihood of rejection of the transplanted organ or tissue, thereby making the patient more vulnerable, susceptible to, or at risk of Aspergillus infection and/or aspergillosis disease. In embodiments, Aspergillus infection and aspergillosis disease are associated with the A. fumigatus fungal pathogen. Patients having other types of diseases and conditions, such as, without limitation, HIV/AIDS, an inflammatory disease, rheumatoid arthritis, or psoriasis, and the like, may also be administered medications having an immune suppressive effect to treat or manage their conditions, and thus, may suffer from, or be at risk of, infection by one or more fungal pathogens, such as A. fumigatus. Non-limiting classes of immune suppressive drugs, agents and medications are described supra and include, for example, corticosteroids, such as prednisone (e.g., DELATSONE, ORASONE); budesonide (ENTOCORT EC), or prednisolone (MLLIPRED) calcineurin inhibitors, such as cyclosporine (NEORAL, SANDIMMUNE, SANGCYA); or tacrolimus (ASTAGRAF XL, ENVARSUS XR, PROGRAF); mTOR inhibitors, such as sirolimus (RAPAMUNE), everolimus (AFINITOR, ZORTRESS); Inosine Monophosphate Dehydrogenase (IMDH) inhibitors, such as azathioprine (AZASAN, IMURAN), leflunomide (ARAVA), mycophenolate (CELLCEPT, MYFORTIC); biologics and monoclonal antibodies or monoclonal antibody-based antibodies or antigen binding fragments thereof, such as abatacept (ORENCIA); adalimumab (HUMIRA); anakinra (KINERET); certolizumab (CIMZIA); etanercept (ENBREL); golimumab (SIMPONI); infliximab (REMICADE); ixekizumab (TALTZ); natalizumab (TYSABRI); rituximab (RITIXAN); secukinumab (COSENTYX); tocilizumab (ACTEMRA); ustekinumab (STELARA); vedolizumab (ENTYVIO), as well as antibodies and immuno-oncology therapeutics, e.g., inhibitors of the PD-1/PD-L1 interaction. In an particular embodiment, the patient is to receive or has received a transplant of an organ selected from kidney, liver, heart, bone marrow, pancreas, lung, gall bladder, bladder, etc. By way of example, an Aspergillus Kexin peptide (or antiserum containing anti-Aspergillus Kexin peptide antibodies) can be administered to the patient who is receiving transplant anti-rejection medication, or other immune suppressive medication, in an effective amount to generate or heighten an immune response against Aspergillus infection and/or aspergillosis in the immune suppressed patient. In an embodiment, the patient receiving immune suppressing drugs can be evaluated and monitored during treatment with immune suppressive drugs for the presence of antibodies (and antibody titers) against the Aspergillus Kexin peptide by employing the methods and kits as described herein. In embodiments, Aspergillus infection and aspergillosis disease are associated with the A. fumigatus fungal pathogen.

In an aspect, an Aspergillus Kexin peptide immunogen (or antiserum containing anti-Aspergillus Kexin peptide antibodies) can be administered to a subject in need thereof in conjunction with another suitable treatment or therapy directed against the Aspergillus fungal pathogen. By way of example, for allergic forms of aspergillosis, such as allergic bronchopulmonary aspergillosis (ABPA) or allergic Aspergillus sinusitis, the antifungal medication itraconazole or corticosteroids may be co-administered to the subject. For IPA, as well as other invasive forms of aspergillosis, e.g., chronic pulmonary aspergillosis and cutaneous aspergillosis, an antifungal medication such as voriconazole may be co-administered. Other antifungal medications that may be co-administered to a subject to treat aspergillosis include lipid amphotericin formulations, posaconazole, isavuconazole, itraconazole, caspofungin, and micafungin. Whenever possible, immunosuppressive medications should be discontinued or decreased in the subject. In some instances of severe aspergillosis, surgery may also be required in addition to the method described herein.

Optionally, an isolated antiserum (e.g., antiserum comprising an anti-Aspergillus Kexin peptide antibodies or antigen binding fragments thereof) may be administered in combination with one or more of any other treatment or therapy, e.g., anti-fungal therapies. For example, an isolated antiserum or immune plasma containing anti-Aspergillus KEX peptide antibodies or antigen binding fragments thereof may be administered in combination with other antibodies or antibody cocktails with anti-fungal activity or in combination with one or more drugs, for examples, one or more drugs having anti-fungal activity (e.g., trimethoprim-sulfamethoxazole, azithromycin-sulfamethoxazole, clarithromycin-sulfamethoxazole, atovaquone, sulfadoxine-pyrimethamine, erythromycin-sulfisoxazole, PS-15, and dapsone-trimethoprim, as well as intravenous pentamidine and clindamycin-primaquine), to provide protective immunity in the recipient against Aspergillus spp., e.g., A. fumigatus, and aspergillosis associated therewith. In an embodiment of any of the foregoing, the Aspergillus Kexin peptide immunogen or an isolated antiserum comprising anti-Aspergillus Kexin peptide antibodies is provided in a pharmaceutically acceptable composition.

In an embodiment of any of the foregoing aspects, the antiserum elicited in or isolated from a subject immunized, inoculated, or administered with an Aspergillus Kexin peptide, e.g., AF.KEX1 peptide as described herein, provides immune protection, including memory immune protection, against infection or aspergillosis disease caused by the Aspergillus fungal pathogen in the subject.

Methods for administering both single and combination therapies (e.g., concurrently or otherwise) are known to those skilled in the art and are described, for example, in Remington's Pharmaceutical Sciences, 12^{th} edition, Edited by E. W. Martin, Mack Publishing Co. In an embodiment, antibodies generated in a subject immunized with Aspergillus Kexin peptide as immunogen, or isolated antiserum containing anti-Aspergillus Kexin peptide antibodies, provides a therapeutic treatment for Aspergillus infection or aspergillosis disease caused by an Aspergillus fungal pathogen. In an embodiment, antibodies generated in a subject immunized with Aspergillus Kexin peptide as immunogen, or isolated antiserum containing anti-Aspergillus Kexin peptide antibodies, provides prophylactic or preventative treatment that protects against the development of aspergillosis disease or reduces the severity of disease and fungal burden caused by an Aspergillus fungal pathogen as described herein. In an embodiment, the Aspergillus Kexin peptide immunogen or the isolated antiserum is in a pharmaceutically acceptable composition. In an embodiment, the Aspergillus fungal pathogen is A. fumigatus.

### Antibodies

As described herein, antisera comprising antibodies that specifically bind the Kexin peptide of an Aspergillus fungal organism, e.g., A. fumigatus Kexin peptide, to provide immune protection against infection and aspergillosis disease, are useful in therapeutic and prophylactic methods. For example, antibodies specifically directed against an Aspergillus Kexin peptide, e.g., the AF.KEX1 peptide as described herein, or an isolated antiserum containing anti-AF.KEX peptide antibodies that target and/or inhibit or neutralize the activity of the Kexin protein of Aspergillus, are particularly useful in the methods of the invention. In particular embodiments, the described methods involving an A. fumigatus KEX peptide such as the AF.KEX1 peptide, as immunogen administered to a subject, generate a robust immune response in the subject in the form of Aspergillus Kexin peptide antibodies, which treat and protect against aspergillosis and the development thereof caused by Aspergillus. In an embodiment, antiserum is obtained or isolated from blood, serum, or plasma of subjects that have generated an immune response.

Methods of preparing antibodies are well known to those of ordinary skill in the science of immunology. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that bind to the target antigen/immunogen and lack the Fc fragment of an intact antibody, clear more rapidly from the circulation, and may have less nonspecific tissue binding than an intact antibody (Wahl et al., J. Nucl. Med. 24:316-325 (1983)). Antibodies may comprise whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062, (1995)), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody, because small molecules of ˜60-100 kDa in size provide faster blood clearance and rapid tissue uptake. See, e.g., Power et al., (Generation of recombinant multimeric antibody fragments for tumor diagnosis and therapy, Methods Mol Biol, 207, 335-50, (2003); and Wu et al., Anti-carcinoembryonic antigen (CEA) diabody for rapid tumor targeting and imaging, Tumor Targeting, 4, 47-58, (1999)).

Various techniques for making and using unconventional antibodies have been described. Bispecific antibodies produced using leucine zippers are described by Kostelny et al. (J. Immunol. 148(5):1547-1553, (1992)). Diabody technology is described by Hollinger et al. (Proc. Natl. Acad. Sci. USA 90:6444-6448, (1993)). Another strategy for making bispecific antibody fragments using single-chain Fv (sFv) diners is described by Gruber et al. (J. Immunol. 152:5368, (1994)). Trispecific antibodies are described by Tutt et al. (J. Immunol. 147:60, (1991)). Single chain Fv polypeptide antibodies include a covalently linked VH:VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, (1988)). See, also, U.S. Pat. Nos. 5,091,513, 5,132,405 and 4,956,778; and U.S. Patent Publication Nos. 20050196754 and 20050196754.

In various embodiments, an antiserum (isolated antiserum) contains anti Aspergillus Kexin peptide (e.g., anti-AF.KEX1 peptide) antibodies or antigen binding fragments thereof which are monoclonal or polyclonal. Hybrid or chimeric antibodies may be produced from anti-Aspergillus Kexin peptide antibodies obtained or isolated from immune serum (antiserum) or immune plasma. In such hybrid or chimeric antibodies, one pair of heavy and light chains is obtained from a first antibody, while the other pair of heavy and light chains is obtained from a different second antibody. Such hybrids or chimeric antibodies may also be formed using humanized heavy and light chains. Methods for isolating antibodies and producing hybrid or chimeric antibodies are known and practiced by those having skill in the art.

In general, intact antibodies are said to contain “Fc” and “Fab” regions. The Fc regions are involved in complement activation and are not involved in antigen binding. An antibody from which the Fc region has been enzymatically cleaved, or which has been produced without the Fc region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies (and immune serum or plasma containing antibodies) can be produced or generated by any of the methods known in the art utilizing polypeptides, or immunogenic fragments thereof, (e.g., an A. fumigatus KEX peptide) as an immunogen. One method of obtaining antibodies is to immunize suitable host animals or subjects with an immunogen and to follow standard procedures for polyclonal or monoclonal antibody production. In brief, immunization will facilitate presentation of the immunogen (or immunogenic fragments of the immunogen) on the cell surface. Immunization of a suitable host can be carried out in several ways. By way of example, nucleic acid sequences encoding an immunogenic Aspergillus Kexin peptide can be provided to the host in a delivery vehicle (or a molecular expression construct) that is taken up by immune cells of the host. The cells will, in turn, process and appropriately express the Aspergillus Kexin peptide in a manner that generates an immunogenic response in the host. In an embodiment, the KEX peptide of Aspergillus fumigatus (e.g., AF.KEX1) may be expressed by the delivery vehicle or expression construct, e.g., E. coli. In other exemplary embodiments, nucleic acid sequences encoding the KEX peptide of Aspergillus fumigatus may be expressed in cells in vitro, and the expressed, recombinant KEX peptide product may be isolated and used as an immunogen to raise anti-Aspergillus KEX peptide antibodies in a subject, as well as to generate an anti-Aspergillus KEX antiserum in an immunized subject.

Alternatively, antibodies against an Aspergillus KEX peptide may be derived from an antibody phage display library. A bacteriophage is capable of infecting and reproducing within bacteria, which can be engineered, when combined with human immunoglobulin (antibody) genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from an immunized host. Antibody purification methods include, without limitation, salt precipitation (for example, with ammonium sulfate), ion exchange chromatography (for example, on a cationic or anionic exchange column, preferably run at neutral pH and eluted with step gradients of increasing ionic strength), gel filtration chromatography (including gel filtration HPLC) and chromatography on affinity resins such as protein A, protein G, hydroxyapatite, or anti-immunoglobulin.

In certain aspects, antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can, in turn, be obtained from the hybridoma that produces the antibody, and then the antibody may be produced synthetically or recombinantly from these nucleic acid sequences. For the production of large amounts of antibody, it is generally more convenient to obtain an ascites fluid. The method of raising ascites fluid generally comprises injecting hybridoma cells into an immunologically naive histocompatible or immunotolerant mammal, especially a mouse. The mammal may be primed for ascites production by prior administration of a suitable composition (e.g., Pristane). Ascites fluid containing antibodies, typically in high concentration, can be obtained from the peritoneal fluid of the animal that harbors the injected hybridoma cells.

Monoclonal antibodies (Mabs) can also be “humanized” by methods known in the art. “Humanized” antibodies are antibodies in which at least part of the sequence has been altered from its initial form to render it more like immunoglobulins derived from a human source. Techniques to humanize antibodies are particularly useful when antibodies are generated in a non-human animal (e.g., mice, rats). Nonlimiting examples of methods for humanizing a murine antibody are provided in U.S. Pat. Nos. 4,816,567, 5,530,101, 5,225,539, 5,585,089, 5,693,762 and 5,859,205.

In an embodiment of the foregoing, one or more antibodies or antigen binding fragments thereof generated against the Aspergillus KEX peptide can be used in a pharmaceutical composition alone or in combination to provide immune protection against disease, such as aspergillosis, or infection caused by an Aspergillus fungal pathogen in a subject in need thereof. Such antibodies may be isolated or purified from an antiserum as described herein, or they may be generated, e.g., by recombinant molecular biology techniques, purified and formulated for pharmaceutical use in a subject in need. Such a formulation of antibodies may have immune protective properties similar to those afforded by an isolated antiserum comprising anti-Aspergillus KEX peptide antibodies as described herein.

### Vaccines

A vaccine is a biological preparation that provides active, acquired immunity (immune protection) in a subject to a particular disease. A vaccine typically contains an agent that resembles a disease-causing pathogenic agent, e.g., a microorganism, a fungus, etc., and is often made from a weakened or killed form of the agent, or from a toxin, or a surface protein or peptide of the agent. After administration of the vaccine to a subject, the agent is expressed and recognized as foreign (or “non-self”) to the subject and stimulates the subject's immune system to mount an immune response (a B cell (antibody) and/or a T cell (cellular) immune response) and to destroy the agent. In addition, cells (e.g., B cells) of the immune system that are exposed to the vaccinating agent retain a memory of the agent, such that the agent is recognized and destroyed by the memory cells upon a later or subsequent encounter. Vaccines can be prophylactic (e.g., to prevent or ameliorate the effects of a future infection by a pathogen), or therapeutic (e.g., to treat disease or infections caused by or associated with pathogens or disease-causing agents upon or after a subject has been infected with or encountered a pathogen).

While many vaccines are prepared from an attenuated version of a pathogen or from inactivated disease-causing organisms, or a suitable part of such pathogens or organisms, such as a toxin, protein/peptide, or deleterious enzyme, the immunogenic antigen to which the immune system responds frequently constitutes a relatively small number of amino acids, such as a peptide (e.g., an A. fumigatus KEX peptide of SEQ ID NO: 2 (AF.KEX1 peptide) derived from Aspergillus Kexin polypeptide as described herein). A Kexin peptide derived from an Aspergillus fungus, e.g., A. fumigatus, may constitute a vaccine. A peptide vaccine is any peptide which serves to immunize an organism. (elicit a therapeutic immune response or a protective immune response, such as an antibody (B cell) response and/or an immune cell (T cell) response in the immunized organism) against a pathogen. In an embodiment, the peptide antigen is the KEX peptide derived from Aspergillus fumigatus, e.g., the AF.KEX1 peptide as described herein. In an embodiment, a vaccine comprising the KEX peptide antigen derived from an Aspergillus fungal organism, such as Aspergillus fumigatus, e.g., AF.KEX1 peptide as described herein, may be used to provide therapeutic treatment or immune protection against aspergillosis following administration to a recipient subject in need.

For non-attenuated vaccines, the peptide sequences that trigger a protective immune response are identified, and synthetic (or recombinantly-produced) versions of the peptides are employed as the vaccine substance. Because they are non-naturally occurring and synthetic, peptide vaccines pose little to no risk of mutation or reversion, and little or no risk of contamination by pathogenic or toxic substances. Moreover, chemical manipulation or modification of the peptide structure may result in increased stability and decreased unwanted side effects or adverse effects that may be associated with a native protein or peptide sequence. Synthetically or recombinantly produced peptide antigens can be readily prepared in large amounts as components of vaccines. Such peptide antigens may also expose parts of a protein antigen that are not recognized by the immune system during a natural infection, possibly resulting from masking or post-translational modifications of proteins.

In an aspect, a therapeutic product, such as a vaccine (or an immunogenic composition) comprising a synthetically (recombinantly) produced peptide, i.e., an Aspergillus Kexin peptide, is provided. Such a product is useful for treating or preventing aspergillosis caused by Aspergillus, e.g., A. fumigatus, after administration (immunization) to a subject. In an embodiment, an Aspergillus Kexin peptide sequence for use in generating an immune response or an antibody response is provided in SEQ ID NO: 2 (AF.KEX1 peptide amino acid sequence), (FIG. 1). In an embodiment, an Aspergillus Kexin peptide sequence for use in generating an immune response or an antibody response is provided in SEQ ID NO: 3. (FIG. 1).

In an embodiment, an Aspergillus Kexin peptide vaccine or immunogenic composition comprising the Aspergillus Kexin peptide, such as the A. fumigatus Kexin peptide described herein, or an immunogenic composition comprising the Aspergillus Kexin peptide, elicits an immune response in an individual following administration (immunization) to an individual in need, resulting in the treatment of aspergillosis disease and protection of the individual from developing aspergillosis. In an embodiment, an Aspergillus Kexin peptide vaccine or immunogenic composition comprising the Aspergillus Kexin peptide, such as the A. fumigatus Kexin peptide, results in the production of antiserum against the Aspergillus Kexin peptide following administration (immunization) to an individual in need. The Aspergillus Kexin peptide immunogen or the isolated antiserum may be used in the treatment or prevention of aspergillosis in an individual in need. In an embodiment, the individual in need has, is susceptible to or at risk of having, aspergillosis, including the different types of aspergillosis as described herein.

### Pharmaceutical Compositions

Featured herein are compositions and methods for treating or preventing aspergillosis disease caused by and/or associated with infection of a subject by an Aspergillus fungal pathogen, e.g., A. fumigatus. In an embodiment, the methods involve administering to a subject in need thereof an effective amount of an Aspergillus KEX peptide immunogen as described herein to treat, prevent, and/or reduce the severity of aspergillosis disease and/or the symptoms thereof caused by Aspergillus in the subject. In an embodiment, the methods involve administering to a subject in need thereof an immunologically effective amount of an isolated antiserum containing antibodies generated against an Aspergillus Kexin peptide, in which the antiserum treats, prevents, and/or reduces the severity of aspergillosis disease and the symptoms thereof caused by Aspergillus in the subject. In an embodiment, the Aspergillus Kexin peptide immunogen or the isolated antiserum is used in a pharmaceutical composition.

Typically, the carrier or excipient for an immunogenic composition or vaccine as described herein is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is affected according to protocols established in the art. Generally, a carrier or excipient is selected to minimize allergic and other undesirable effects, and to suit the particular route of administration, e.g., subcutaneous, intramuscular, intranasal, and the like. Such methods also include administering an adjuvant, such as an oil-in-water emulsion, a saponin, a cholesterol, a phospholipid, a CpG, a polysaccharide, variants thereof, and a combination thereof, with the composition of the invention. Optionally, a formulation for prophylactic administration also contains one or more adjuvants for enhancing the immune response to an antigen or immunogen, such as an Aspergillus (e.g., A. fumigatus) KEX peptide antigen or immunogen. Suitable adjuvants include, without limitation, complete Freund's adjuvant, incomplete Freund's adjuvant, saponin, mineral gels such as aluminum hydroxide, alum, surface active substances such as lysolecithin, pluronic polyols, polyanions, peptides, oil or hydrocarbon emulsions, bacille Calmette-Guerin (BCG), Corynebacterium parvum, TITERMAX, and the synthetic adjuvants QS-21 and MF59 (Novartis). By way of example, TITERMAX adjuvants produce both humoral and cellular immune responses and comprise a water-in-oil emulsion, including squalene (a metabolizable oil), an emulsifier (e.g., sorbitan monooleate or sorbitan monooleate 80), a block copolymer (e.g., CRL89-41 or CRL-8300, Sigma-Aldrich) and microparticulate silica (Stills, J. F., 2005, ILAR Journal, 46(3):280-293). In an embodiment, the isolated antiserum is used in a pharmaceutical composition.

The administration of an Aspergillus Kexin peptide immunogen may be carried out by any suitable means that results in a concentration of the immunogen or therapeutic that, combined with other components, if desired, is effective in ameliorating, reducing, eliminating, abating, protecting against, treating, or stabilizing aspergillosis disease and/or its symptoms in a subject. The immunogen or therapeutic may be administered systemically, for example, formulated in a pharmaceutically-acceptable composition or buffer such as physiological saline. Preferable routes of administration include, for example, subcutaneous, intravenous, intraperitoneal, intramuscular, intrathecal, or intradermal injections that provide continuous, sustained levels of the immunogen or therapeutic in the subject. The amount of the immunogen or therapeutic to be administered varies depending upon the manner of administration, the age and body weight of the subject, and with the clinical symptoms of aspergillosis in the subject. Generally, amounts will be in the range of those used for other agents used in the treatment of aspergillosis, although in certain instances, lower amounts may be suitable because of the increased range of protection and treatment afforded by the immunogen or therapeutic. A composition is administered at a dosage or effective amount that ameliorates, decreases, diminishes, abates, alleviates, or eliminations the effects of aspergillosis disease or the symptoms thereof as determined by a method known to one skilled in the art. In an embodiment, Aspergillus Kexin peptide immunogen, or anti-Aspergillus Kexin peptide antiserum (isolated antiserum) is administered or provided to a recipient subject at or near a site of the infection or colonization by the Aspergillus pathogenic organism.

In embodiments, a therapeutic or prophylactic treatment agent may be contained in any appropriate amount in any suitable carrier substance, and is generally present in an amount of 1-95% by weight of the total weight of the composition. The composition may be provided in a dosage form that is suitable for parenteral (e.g., subcutaneous, intravenous, intramuscular, intrathecal, or intraperitoneal) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions may in some cases be formulated to release the active agent substantially immediately upon administration or at any predetermined time or time period after administration. The latter types of compositions are generally known as controlled release formulations, which include (i) formulations that create a substantially constant concentration of a therapeutic agent or drug within the body over an extended period of time; (ii) formulations that after a predetermined lag time create a substantially constant concentration of a therapeutic agent or drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the lungs; (v) formulations that allow for convenient dosing, such that doses are administered, for example, once every one or two weeks; and (vi) formulations that target aspergillosis using carriers or chemical derivatives to deliver the therapeutic agent or drug to a particular cell type, e.g., the lungs or lung cells and tissue. For some applications, controlled release formulations obviate the need for frequent dosing during the day to sustain a therapeutic level in plasma, serum, or blood. In an embodiment, an isolated antiserum may be formulated with one or more additional components for administration to a subject.

Any of a number of strategies can be pursued in order to obtain controlled release in which the rate of release outweighs the rate of metabolism of the therapeutic agent or drug in question. In one example, controlled release is obtained by appropriate selection of various formulation parameters and ingredients, including, e.g., various types of controlled release compositions and coatings. Thus, the therapeutic agent or drug may be formulated with appropriate excipients into a pharmaceutical composition that, upon administration, releases the therapeutic agent or drug in a controlled manner. Examples include single or multiple unit tablet or capsule compositions, oil solutions, suspensions, emulsions, microcapsules, microspheres, molecular complexes, nanoparticles, patches, and liposomes.

A pharmaceutical composition may be administered parenterally by injection, infusion or implantation (subcutaneous, intravenous, intramuscular, intraperitoneal, intrathecal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, noted supra.

Compositions for parenteral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a disease or dysfunction, such as pulmonary disease or dysfunction, the composition may include suitable parenterally acceptable carriers and/or excipients. In some cases, an active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, a pharmaceutical composition comprising an active therapeutic (e.g., an Aspergillus Kexin peptide or an isolated anti-Aspergillus Kexin peptide antiserum as described herein) is formulated for intravenous delivery, e.g., intravenous, injection, or intrathecal delivery. To prepare such a composition, the suitable therapeutic(s) are dissolved or suspended in a parenterally acceptable liquid vehicle, excipient, or solvent. Among acceptable vehicles and solvents that may be employed are, for example, water; water adjusted to a suitable pH by the addition of an appropriate amount of hydrochloric acid, sodium hydroxide or a suitable buffer; 1,3-butanediol; Ringer's solution; and isotonic sodium chloride solution and dextrose solution. The aqueous formulation may also contain one or more preservatives (e.g., methyl, ethyl or n-propyl p-hydroxybenzoate). In cases in which one of the agents is only sparingly or slightly soluble in water, a dissolution enhancing or solubilizing agent can be added, or the solvent may include 10-60% w/w of propylene glycol or the like.

### Kits

In another embodiment, kits and compositions are provided that advantageously provide reagents for treating or protecting a subject in need thereof against aspergillosis associated with Aspergillus infection. In an embodiment, the kit contains an Aspergillus Kexin peptide immunogen for delivery to a subject. In an embodiment, the Aspergillus Kexin peptide comprises or consists of SEQ ID NO: 2 (AF.KEX1 peptide). In another embodiment, the Aspergillus Kexin peptide comprises or consists of SEQ ID NO: 3. In an embodiment, the subject is a human patient. In an embodiment, the patient is immunosuppressed and thus may be at higher risk for infection by an Aspergillus fungal organism, e.g., A. fumigatus. In an embodiment, the patient is immunocompromised and thus may be at higher risk for infection by an Aspergillus fungal organism, e.g., A. fumigatus. In an embodiment, the patient has undergone a transplant, e.g., an organ or tissue transplant, or is to undergo a transplant, and thus may be at higher risk for infection by an Aspergillus fungal organism. In an embodiment, the transplant patient, or the patient to undergo a transplant, is immunosuppressed and/or is otherwise treated with drugs to reduce the likelihood of rejection of the transplanted organ or tissue, thereby making the patient more vulnerable or susceptible to infection and/or aspergillosis disease caused by an Aspergillus fungal pathogen. In an embodiment, the patient has received, or is to receive, a transplant of an organ selected from kidney, liver, heart, bone marrow, pancreas, lung, etc.

A kit may further comprise reagents that allow for assessing, measuring, evaluating or detecting antibodies generated in an immunized subject and directed against the Aspergillus Kexin peptide as immunogen. Such antibodies may be contained in a biological sample obtained from a subject undergoing testing, assessment, or evaluation using the kit. In particular, the biological sample may be a blood, serum, plasma, bronchiolar lavage, pulmonary lavage, or a lung cell or tissue sample obtained from a subject.

In embodiments, the kit may contain instructions for use and may include at least one of the following: description of the Aspergillus Kexin peptide immunogen; dosage schedule and administration for treatment or prevention of aspergillosis and/or the symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

The following examples are put forth so as to provide those of ordinary skill in the art with a complete disclosure and description of how to make and use the described aspects and embodiments, e.g., immunogenic products, compositions and methods, and are not intended to limit the scope of what the inventors regard as their invention.

### Example 1: Aspergillus Kexin (KEX1) Peptide

A recombinant Kexin peptide comprising a 90 amino acid sequence of Aspergillus fumigatus Kexin protein (Accession no. XM746441) was generated by cloning the corresponding DNA sequence into an E. coli expression vector and producing the 90-amino acid recombinant protein (called AF.KEX1 peptide herein, FIG. 1). AF.KEX1, (PG90), was shown to be a target of humoral immune responses during Aspergillus infection, and anti-Aspergillus KEX peptide antibodies were present in antiserum obtained from animals (Aspergillus-infected mice) having Aspergillus infection/asthma.

### Example 2: Procedures for the Purification of A. fumigatus Recombinant KEX Protein

A protocol used for expressing and purifying a recombinantly produced A. fumigatus KEX1 peptide (pET28b vector), (Millipore-Sigma, US), is provided. The protocol is employed for the Aspergillus KEX1 peptide expressing construct in BL21(DE3)/BL21(DE3)pLys E. coli bacterial cells. The peptide may also be histidine tagged.

### Materials and Equipment

A. LB (Lysogeny Broth) growth medium with kanamycin (40 μg/mL), typically in a 1 L volume, pH to 7.5. 10 g NaCl, 5 g Yeast Extract and 10 g Tryptone Peptone are admixed; pH to 7.5, and the volume is brought to 1 L with distilled/deionized H₂O.
B. 1M IPTG solution (0.2 μm sterile filter, 200 μl aliquots).
C. A stock solution of 1M Sodium Phosphate (pH 7.4) was prepared and stored at 4° C.
D. Prepare a stock solution of 5M sodium chloride (NaCl).
E. Prepare 300 mL of Wash Buffer (no imidazole) containing 50 mM Sodium Phosphate, 300 mM NaCl, pH 7.4. Store at 4° C.
F. Prepare 50 mL of 1M imidazole solution in Wash Buffer (50 mM Sodium Phosphate, 300 mM NaCl, pH 7.4) and 0.2 μm sterile filter. Store at 4° C.
G. Prepare 250 mL of Wash buffer containing 10 mM imidazole. Supplement remaining 250 ml of Wash Buffer with appropriate 1M imidazole stock. Store at 4° C. Both solutions are sterile filtered
H. Prepare 250 mL of Denaturing Extraction Buffer (0.2 μm sterile filter) containing 50 mM Sodium Phosphate, 300 mM NaCl, 10 mM imidazole, 6M Guanidine-HCl, pH 7.4. Store at 4° C.
I. Talon metal affinity resin (Clontech P #635503). Alternative: HisPur Cobalt Resin.
J. Disposable 5 mL polypropylene column (Thermo P #29922).
K. His-tag protease inhibitor cocktail (PIC) (Sigma P #8849).

### L. Bio-Rad Protein Assay Dye Reagent Concentrate (Bio Rad Catalog #500-0006).

M. SPECTRA™ Multicolor Ladder-Broad range stained (Thermo P #22634).

### N. Laemmli Buffer (6×).

O. Hoefer gel casting system (model SE250).
The procedure used is as follows:
A. Culture and induce protein expression in E. coli:
a. Streak out monkey KEX1 pET28b onto LB KAN agar plates. Incubate at 37° C. overnight (0/N) and select for single colonies.
b. Inoculate a single colony into 10 mL liquid LB KAN 40 (allowing ˜1:5 liquid to air ratio) and place on shaker 0/N at 37° C. O/N to allow growth of bacteria in culture.
c. Following overnight incubation, dilute culture 1:50-1:200 into liquid LB KAN 40 and leave at 37° C. on shaker.
d. Grow cultures to an OD₆₀₀=0.5 and then add 0.5 mM IPTG to induce expression for 4 hours at 37° C. on shaker.
e. Harvest cells in 250 mL Oakridge tubes and centrifuge at 6,000×g and 4° C. for 25 minutes (use SS-34 or SLA-1500 rotor).
f. Decant supernatant and resuspend cells in ˜200 mL 1×PBS to wash out residual medium. Pour off supernatant.
g. Store cell pellets at −80° C. until time of use. Do not store E. coli pellets for longer than two weeks prior to protein extraction.
   B. Protein purification using Talon metal affinity resin:
a. Thaw pellet on ice and re-suspend cell pellet in 10 mL Denaturing Extraction Buffer+200 μL PIC.
b. Solubilize protein by incubating at room temperature (RT) for 2 hours on nutator.
c. Clarify lysate as follows:
   i. Centrifuge suspension at 10,000×g and 4° C. for 20 minutes (use SS-34 rotor).
   ii. Collect supernatant.
   iii. Aliquot supernatant into 1.5 mL centrifuge tubes in a volume of approximately 1 mL/tube
   iv. Centrifuge at 16,000-17,000 g for 20 minutes at 4° C.
   v. Collect clarified supernatant. If lysate is not completely clear, transfer to new test tube and re-centrifuge.
d. Collect supernatant and keep on ice until Talon resin is prepared.
e. Prepare polypropylene elution column by suspending column in the upright position; adding a few drops of wash buffer to a porous disc, then using the reverse end of a Pasteur pipette to depress the disc evenly to the bottom of the column.
f. Prepare Talon resin by resuspending the Talon resin by gently shaking; adding 3.5 mL of resin to a 15 mL conical tube, and centrifuging for 5 minutes at 500 g. The ethanol layer is carefully removed without disturbing the resin. Add 10 mL of dH₂O to wash the resin and recentrifuged for 5 minutes at 500 g. Carefully remove the supernatant and discard. Equilibrate the resin in 10 mL of Extraction Buffer and centrifuge for 5 minutes at 500 g. Carefully remove the supernatant and discard.
g. Batch bind the lysate with equilibrated resin for 1 hour at 4° C. (on nutator in the cold room).
h. While the lysate is binding, prepare a disposable 5 mL polypropylene column (Thermo P #29922) as follows:
   i. Soak filter/biscuit/disc in Denaturing Extraction Buffer to ensure no air is trapped.
   ii. Cap column and fill with Denaturing Extraction Buffer.
   iii. Use reverse end of a Pasteur pipette to depress disc evenly to the bottom of the column.
   iv. Drain all but 500 ml of buffer from column and re-cap.
i. Add lysate and the resin to column and allow to drain.
j. Wash resin with at least 20 column volumes:
   i. Wash with 10-15 column volumes of Denaturing Extraction Buffer (approximately 20 mL).
   ii. Wash with 10-15 column volumes of Wash Buffer (approximately 20 mL).
      If purifying an insoluble protein, e.g., A. fumigatus KEX1 peptide, the below Steps k and 1 were omitted, and the purification protocol was resumed at Step m.
k. Elute in 1.5 ml fractions with increasing imidazole concentration in Wash buffer (native, no guanidine).
   i. Most contaminants elute off at 100 mM, but most of the protein elutes off at 150 mM.
   ii. Suggested imidazole concentrations-75 mM, 100 mM, 125 mM, 150 mM, 175 mM, 200 mM (1), 200 mM (2), 200 mM (3), 250 mM.
   l. Add 204, PIC to each fraction of interest and store @ 4° C.
   m. Cap the bottom of the column and resuspend the resin in 1 mL of 1% sodium dodecyl sulfate (SDS) by adding the solution directly to the resin in the column and triturating the mixture with a pipette.
   n. Aliquot the resuspended resin into 1.5 mL tubes labeled with the corresponding lot of protein. Approximately 4 tubes are used. All of the resin chunks must be collected, as the protein product of interest is bound to the resin.
   o. Place the aliquots on the 95° C. heating block for 15 minutes.
   p. Centrifuge the aliquots at 500 g for 5 minutes. While the aliquots are in the centrifuge, label fresh microcentrifuge tubes with the protein lot and date. Once centrifugation is complete, aliquot the supernatant into the corresponding tubes.
   q. Repeat Steps M through P for 4 more times (a total of 5 boils). [Note: Instead of filling the resin column with 1 mL of 1% SDS, the remaining pelleted microcentrifuge tubes will be filled with 1 mL of 1% SDS.]
   r. Prepare the acidified acetone/methanol mix by combining acetone with HCl and methanol at a 1:1 ratio (120 μl Acetone with 10 μl HCl and 120 μl Methanol, 1 mM final concentration). Store at −20° C.
   s. Add a 1:4 mix of each boil and acetone mix and place at −20° C. O/N.
   t. Centrifuge the samples at maximum speed for 15 minutes at 4° C. u. Discard the supernatant and avoid disturbing the pellet.
   v. Allow the pellet to dry for a minimum of one hour. Prepare an ice bucket.
   w. After the pellets are completely dry, resuspend the samples in 50 μL of PBS and keep on ice.
      i. Consolidate the aliquots by boil. Because the later boils are less concentrated, the volume of PBS for these later boils are adjusted accordingly.
      ii. Record the total volume for each boil.
         C. Quantification of protein in elution fractions (BioRad Protein Assay Catalog #500-0006). This must be performed on the same day as gel electrophoresis is carried out.
a. The Bio-Rad Protein Assay was carried out according to the manufacturer's protocol.
b. Albumin Standard ThermoFisher #23209 is removed and protein standards are prepared: 0.5-0.05 mg/ml with 2-step dilution. The linear range of this microtiter assay is 0.05-0.5 mg/ml. Commercial standards are necessary for reproducible results between purifications. Standards are not prepared from BSA.
c. Elution fractions and standards are measured in duplicate.
d. Samples are diluted with water and 6× Laemmli Buffer:
   i. Coomassie staining (10 μg/well or up to 40 μl).
   ii. Western Blotting (5 μg/well or up to 40 μl).
   iii. Add 8 μl of 6× Laemmli Buffer (5% 2-mercaptoethanol) in 40 μl sample. Diluted samples should be ˜48 μl. (Maximum volume if wells are 50 μl with 9-well 1.5 mm combs).
   iv. Boil samples for 5 minutes at 95° C.
   v. Centrifuge at full speed for 5 minutes to pellet debris.
   vi. Store denatured samples at −20° C. until next day.

### D. Gel Electrophoresis:

a. Prepare 15% resolving/4% stacking polyacrylamide gels according to manufacturer's protocol.
b. Assemble running apparatus and fill inner chamber and bottom tray with 1×SDS-PAGE running buffer. Remove comb and wash out wells. Do not re-use inner chamber buffer.
c. Load 10 μL of the Broad Range stained (P #26634) SPECTRA™ Multicolor Ladder diluted in 1× Laemmli buffer. Gels run straighter if all samples/standard are the same volume and if all wells are full.
d. Load denatured samples.
e. Let the gel run at constant voltage, —80-120 volts, for 1.5-2.5 hours until the dye front runs off the bottom of the gel. (Note: For straight gels, the voltage may be decreased; however, when using hand-cast gels, the voltage is never increased once gel electrophoresis has begun).
f. After gel electrophoresis has ended, remove stacking gel with scraper.
g. Wash gels with dH₂O before Coomassie Staining or Western Blotting.
   E. Coomassie Staining (Measures purity of fractions)
a. Add ˜25 mL of Coomassie Blue stain. Microwave covered for 1 minute.
b. Cool by rocking for 15 minutes at RT.
c. Discard stain and wash with dH₂O until water is clear.
d. Add ˜25 mL of Destain to gel and a Kim Wipe (absorbent tissue). Rock on nutator 0/N at RT. Kim Wipe will absorb excess Coomassie stain so that destain will not need to be changed overnight.
   F. Western blotting is performed on samples using standard protocols in the art.

### Example 3: Protocol for Generating Tagged Recombinant A. fumigatus KEX Proteins

A polynucleotide sequence encoding a Kexin peptide of Aspergillus fumigatus (FIG. 1) was synthesized and inserted into the expression vector pMAL-c4× using BamHI and HindIII restriction sites (GenScript). Each insert contained an N-terminal tobacco etch virus (TEV) cleavage site, a maltose binding protein (MBP) tag, and an additional transcriptional start site (ATG) 5′ to the conserved KEX sequences followed by two stop codons (AAG, CTT, ochre and opal, respectively). Plasmids were transformed into Escherichia coli BL21 (DE3) cells and plated on LB agar supplemented with 100 μg/ml ampicillin to select for transformed clones. For the preparation of recombinant KEX protein, expression hosts were grown overnight with shaking at 37° C. in Luria broth (LB) supplemented with 100 μg/mL ampicillin and then sub-cultured 1:20 for 2 hours at 37° C., and protein expression was induced by the addition of 1 mM IPTG, with further incubation for 4 hours at 37° C. Expression of recombinant maltose binding protein (MBP)-tagged fusion proteins was confirmed by Western blotting using commercially available anti-MBP sera (New England BioLabs).

Harvested cells were then resuspended in 20 mM Tris-HCl, 300 mM NaCl, 1 mM EDTA, pH 8.0 with protease inhibitor cocktail (Sigma) and lysed through cell disruption. Supernatants were collected following centrifugation at 20,000×g for 30 minutes at 4° C. MBP-tagged proteins were purified by affinity chromatography using amylose resin (New England BioLabs). To cleave the ˜42.5 kDa N-terminal MBP tag from recombinant KEX proteins, purified proteins were incubated with enhanced TEV protease (AcTEV, Invitrogen) at a 1:100 protease to target ratio for ˜4 hours at 30° C. in a buffer containing 20 mM Tris-HCl, 300 mM NaCl, 1 mM EDTA, pH 8.0. The TEV protease cleaved the N-terminal maltose binding protein (MBP) affinity tag (Aspergillus KEX-MBP+TEV) and yielded a reagent suitable for use in immunoblotting studies following resolution of recombinant proteins by 15% SDS-PAGE. Following TEV cleavage, each recombinant protein contained an additional N-terminal glycine and methionine due to the TEV cleavage motif and the added transcriptional start site, respectively.

### Example 4: A. fumigatus KEX Peptide Enzyme Linked Immunosorbent Assay (ELISA)

This example describes a protocol for performing an ELISA immunoassay utilizing a recombinantly produced (pET28b vector), and an A. fumigatus KEX protein (peptide) that is histidine tagged and purified as described above in Example 2. The ELISA was conducted to detect (and quantify) the presence of anti-Aspergillus KEX peptide (e.g., AF.KEX1 peptide) antibodies in a sample, e.g., blood, plasma, serum, bronchoalveolar lavage, pulmonary lavage, or other biological fluid sample. The anti-KEX peptide antibodies to be detected (and quantified) are directed against, reactive with and/or bind to the KEX peptide of Aspergillus spp., e.g., A. fumigatus, e.g., the 90-mer (AF.KEX1 peptide, SEQ ID NO: 2) or the 88-mer KEX peptide of A. fumigatus (SEQ ID NO: 3) as described herein.

### Materials and Equipment

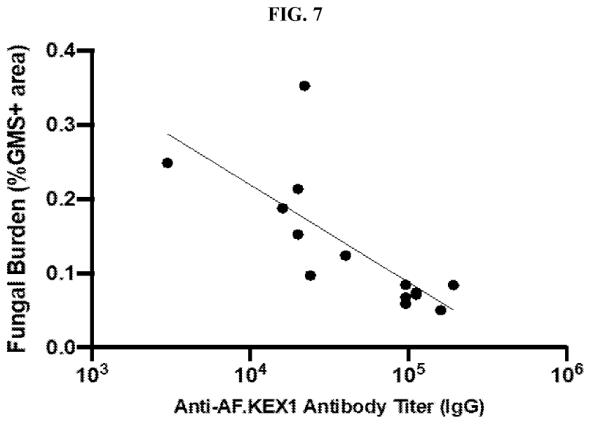

A. KEX protein, which may be purified as described in Example 2
B. 1×PBS
C. Immulon high-binding (4HBX) Flat bottom microtiter plates (Thermo #3855)
D. Blocking buffer: 5% skim milk in 1×PBS
E. Wash buffer: 1× Phosphate-buffered Saline (PBS)+0.05% Tween-20
F. Secondary Antibody: Goat anti-human immunoglobulin-conjugated horseradish peroxidase (1:10,000 for IgG; Sigma-Aldrich).
G. Normal human plasma (Atlanta Biologicals, Inc., Lawrenceville, Ga.). Negative/normal control plasma with undetectable absorbance at OD₄₅₀ (i.e., equal to or less than dilution buffer alone) in KEX-ELISA at a dilution of 1:100 is used as negative controls.
H. Substrate: 3,3′,5,5′-Tetramethylbenzidine (TMB) peroxidase substrate (such as SureBlue TMB substrate, 1-component; KPL, Inc.)
I. Stop solution: 1 M H₂SO₄
J. Adhesive sealing film for microplates (Plate sealers) (such as SealPlate non-sterile films from Excel Scientific, cat #100-SEAL-PLT)
K. 96-well plate reader (any system capable of reading OD at a wavelength of 450 nm). The procedure used for performing the ELISA is as follows:
A. Coating/blocking ELISA plates with KEX protein:
   a. Prepare mkKEX protein in 1×PBS at 5 ug/mL. Add 50 μL of diluted KEX per well of Immulon 4HBX flat-bottom ELISA plates. Cover plates tightly with Parafilm or plate sealers and incubate 0/N at 4° C.
   b. Following overnight incubation, remove buffer by flicking into sink or bucket and tap plate onto absorbant pad or paper towels to remove excess. Wash plates 2× with wash buffer (PBS 0.05% Tween-20) (2004, wash buffer per well for each wash, flicking and tapping plate between washes).
   c. Add 100 μL of blocking buffer (5% milk/PBS) to each well and incubate for 1 hour at 37° C.
   d. Empty plates, wash 2× with wash buffer. The plates can be sealed and frozen at −20° C. at this step, until ready for use.
B. Handling of plasma or other infectious fluids (e.g., bronchoalveolar lavage (BAL) fluid supernatant, etc.)—First-time use.
   a. Remove plasma aliquot from −80° C. freezer.
   b. Option 1: Heat-inactivate entire aliquot at 56° C. for 30 minutes. Option 2: If heat inactivation of the plasma sample would be detrimental to other potential uses, thaw sample at 4° C. or on ice. Remove an aliquot (˜100 transfer to a new tube, and heat inactivate (30 min, 56° C.). Return the remaining sample to the −80° C. freezer, noting that it has been thawed 1×.
   c. Centrifuge sample at >10,000 g for 1-2 minutes to pellet aggregates prior to use.
   d. To prevent contamination in storage, add ˜0.01 to 0.02% NaN₃. Store sample aliquot for up to 6 months at 4° C. For subsequent assays, no further heat inactivation is needed; however, the sample should be centrifuged briefly prior to each use.
C. ELISA for endpoint titer determination (plasma):
   a. Dilute plasma 1:100 in blocking buffer. Add 50 μL of diluted plasma and make serial 2× (or 4×, if needed) dilutions directly in the plate (final volume in each well should be 50 μL) for generation of endpoint titers (see, FIGS. 7A and 7B). Perform assay in duplicate; set up enough plates for all isotypes of interest, e.g., if there are 10 samples and endpoint titers are to be generated for both IgG and IgM-KEX antibodies, this would require setting up 4 plates (duplicate plates for both IgG and IgM). Include a negative/normal control on each plate. Cover plates with plate sealers and incubate 0/N at 4° C.
   b. Empty plate (flicking and tapping), wash 4× w/wash buffer.
   c. Add 50 μL of secondary antibody (diluted in block) to each well (see appropriate dilutions under Materials and Equipment above). Incubate 1 hour at 37° C.
   d. Empty the plate and wash 6× with wash buffer.
   e. Add 100 μL of TMB to each well, protect from light and incubate for 30 minutes at 37° C.
   f. Add 25-50 μL of stop solution (1 M H₂SO₄) to each well.
   g. Read OD of plates (on any standard plate reader) at 450 nm within 20 minutes of adding stop solution.

### Example 5: Induction of an Immune Response Against an A. fumigatus Kexin Peptide Immunogen and Protection Against Invasive Pulmonary Aspergillosis (IPA) in a Mouse Model of IPA

### Humoral Immune Responses to AF.KEX1 Peptide as Immunogen

Antibodies that recognize and bind to the AF.KEX1 peptide were generated during A. fumigatus challenge and were detected by Western blot (FIGS. 3A and 3B) using plasma from a single mouse prior to and following Af293 challenge. The immunogenicity of the AF.KEX1 peptide immunogen was evaluated by administration to animals (e.g., by sc or iv immunization) of recombinant AF.KEX1 peptide in conjunction with TITERMAX or of PBS and TITERMAX (controls), e.g., according to the schedule shown in FIG. 4A. Anti-AF.KEX1 peptide antibody titers significantly increased following injection of animals with AF.KEX1 peptide; antibody titers peaked at 28 days post immunization, while no significant change was observed in the sham-immunized cohorts (PBS and TITERMAX, FIG. 4B).

The potential of increasing or enhancing the immune response with an additional boost of the AF.KEX1 peptide immunogen was also evaluated. It was found that while a higher peak antibody titer was achieved following the boost, no significant difference in anti-AF.KEX1 peptide antibody titer was observed at 28 days post-boost (day 56) compared with the antibody titer observed at 28 days following the initial immunization (FIGS. 4C and 4E). These results were further corroborated by Western blot of AF.KEX1 peptide with antiserum, which showed that antibody recognition was greatly increased in animals that had been immunized with the recombinant AF.KEX1 peptide compared to the sham-immunized control cohort (FIG. 4D).

### AF.KEX1 Immunization Protects Against IPA in Immunosuppressed CF-1 Mice

Following immunization, 15 AF.KEX1 peptide-immunized mice and 17 sham-immunized control mice were immunosuppressed for six days as described herein before the mice were intranasally challenged with 5×10⁶ A. fumigatus (Af293) conidia (FIG. 5A). Following challenges, the mice were observed twice daily for signs of aspergillosis, including weight loss and drops in temperature. One of the AF.KEX1 peptide-immunized mouse was censured from analysis due to mortality unrelated to the study. Over the observation period, aspergillosis was observed to develop in 4 of the sham-immunized cohort animals, while no mortality was observed in animals immunized with the AF.KEX1 peptide immunogen (p=0.0487), (FIG. 5B). These results were further supported by analysis of fungal burden in the study animals. Fungal burden analysis was conducted using both GMS staining and qPCR. In both types of analyses, fungal burden was significantly reduced in animals immunized with AF.KEX1 peptide compared to the animals in the sham-immunized cohort (FIGS. 6A-6D). In addition, it was determined that a correlation existed between the peak antibody titer achieved following immunization with the AF.KEX1 peptide and TITERMAX, and the terminal fungal burden as determined by GMS stain quantification (FIG. 7).

### Materials and Methods

### Vaccine Construction and Purification

A 90 amino acid peptide fragment of Aspergillus fumigatus KEXB protein (AF.KEX1 peptide, reference sequence XP_751534.1), SEQ ID NO: 2, (FIG. 1), was cloned into the pET28b(+) expression vector (Novagen) in Escherichia coli BL21(DE3) pLysS (ThermoFisher, Scientific) and was purified by affinity chromatography, e.g., as described in Example 2 above. The resin-bound protein was boiled in 1% SDS for 15 minutes and precipitated overnight at −20° C., 1:4 in acidified acetone/methanol. The purified AF.KEX1 peptide was used for immunization and enzyme-linked immunosorbent assay (ELISA). An 88 amino acid peptide fragment, e.g., SEQ ID NO: 3, of Aspergillus fumigatus KEXB protein (reference sequence XP_751534.1) can also be employed as an immunogen following cloning into the pET28b(+) expression vector in Escherichia coli BL21(DE3) pLysS and purified as described herein.

### Study Design

Thirty-two CF-1 mice were purchased from Charles River Laboratories. Studies were approved by the Institutional Animal Care and Use Committee (IACUC) of the University of Georgia. Mice were assigned to one of four cohorts for immunization and challenge. Blood was collected from the animals at baseline and 14, 21, and 28 days following each immunization. Plasma samples were stored at −80° C.

### Immunization and Immunosuppression

Five (5) mice were immunized subcutaneously at the base of the tail with 50 μg AF.KEX1 peptide prepared 1:1 with TITERMAX adjuvant according the adjuvant guidelines (Group 1). An additional 7 mice were sham-immunized with phosphate buffered saline (PBS) and TITERMAX (Group 2). Ten (1) additional mice were immunized and boosted with 50 μg AF.KEX1 peptide prepared 1:1 with TITERMAX to evaluate the need for a boosting immunization (Group 3). A final group of 10 animals received no immunization (Group 4).

Twenty-eight (28) days following the final immunization, all mice were placed on an immunosuppressive regimen. Each mouse received 2.5 mg cortisone acetate in PBS with 0.5% methylcellulose and 0.01% Tween-80 injected subcutaneously. This regimen was administered for six days, during which time trimethoprim sulfamethoxazole was added to the drinking water to control secondary infections.

### ELISA Immunoassay

Microtiter plates (Immunolon 4HBX; Thermo Fisher Scientific) were coated with purified AF.KEX1 peptide at 5 μg/ml in PBS. Heat-inactivated plasma samples were diluted 1:100 in blocking buffer (PBS with 5% nonfat milk) and 1:2 serial dilutions were made to determine endpoint titers. Goat anti-mouse immunoglobulin conjugated horseradish peroxidase was used for detection, and plates were developed with TMB. Naïve (uninfected, A. fumigatus-negative by antibody titer) mouse plasma was used as a negative control.

Aspergillus fumigatus Challenge and Monitoring

A. fumigatus Af293 conidia were maintained on solid 1% glucose minimal medium for 72 hours, harvested in 0.01% Tween-20, counted using a hemocytometer, and then diluted in PBS. Mice were inoculated with 5×10⁶ conidia in 40 μl PBS via intranasal inoculation (A. fumigatus challenge). Following challenge, mice were monitored twice daily for changes in weight and temperature. If weight loss greater than 20 percent or body temperature below 29° C. were recorded, the animals were humanely sacrificed. At six days following challenge, all remaining animals were sacrificed; the lungs of these animals were collected for analysis.

### Fungal Burden

Following sacrifice, the right lungs of all animals were stored in formalin, and the left lungs were frozen in liquid nitrogen. The fixed lung tissue was embedded in paraffin, cut, and stained with Gomori's modified methanamine silver stain. Images of five distinct fields were taken and fungal burden was quantified according to the guidelines provided by Stolz et al., 2018, “Histological Quantification to Determine Lung Fungal Burden in Experimental Aspergillosis,” Journal of visualized experiments: JoVE, (133), 57155. The frozen lung tissue was lyophilized and homogenized with 0.5 mm disruption glass beads (RPI, catalog #9831) and three 3 mm steel beads using GeneGrinder at 1750 rpm for 30 seconds. Total DNA was extracted using a modified CTAB protocol as described by Pitkin, J. W. et al. (1996, Microbiology, 142:1557-1565). RNAse treated DNA was used for qPCR as previously described (Johnson, G. L. et al., 2012, “A MIQE-Compliant Real-Time PCR Assay for Aspergillus Detection,” PLoS One, 7(7):e40022. Doi:10.1371/journal.pone.0040022) on a Mx3005P real-time PCR system (Agilent Technologies, Inc. Santa Clara, Calif., USA). A reaction volume of 50 μl with 5 μl of diluted DNA and 25 μl of 2× PowerUp SYBR Green Master Mix (Applied Biosystems) was used. Thermal cycling conditions were 95° C. for 10 minutes; 40 cycles of 95° C. for 30 seconds and 60° C. for 1 minute. Melt curve analysis was performed at the end of the qPCR reactions to check for primer specificity. Controls without a template were also analyzed by qPCR.

### Statistical Analysis

All statistical analyses were performed using GraphPad Prism (GraphPad Software, La Jolla, Calif.). Differences in post-immunization anti-AF.KEX1 peptide antibody titer were analyzed using Mann-Whitney U tests. A Mantel-Cox test was used to analyze the survival curves following A. fumigatus challenge. Differences in fungal burden by both GMS staining and qPCR were analyzed by Mann-Whitney U tests. The correlation between anti-AF.KEX1 peptide antibody titer and terminal fungal burden was determined by Spearman correlation.

### Example 6: Immunogenicity and Protective Efficacy of the AF-KEX1 Immunogen as a Vaccine in Immunosuppressed Subjects

In this Example, the immunogenicity and protective efficacy of the AF-KEX1 peptide immunogen administered as a vaccine (e.g., prior to Aspergillus challenge) in immunosuppressed subjects was further demonstrated. It was determined that the immunization protocol using the AF-KEX1 peptide immunogen was effective in a murine model of immunosuppression. A combination immunosuppressive regimen, which included hydrocortisone and FK506 (Tacrolimus), i.e., an inhibitor of antigen-specific T cell activation and differentiation, were administered to mice. Such an administration regimen models a more comprehensive immunosuppressive regimen that is used to mitigate graft rejection in organ transplant recipients.

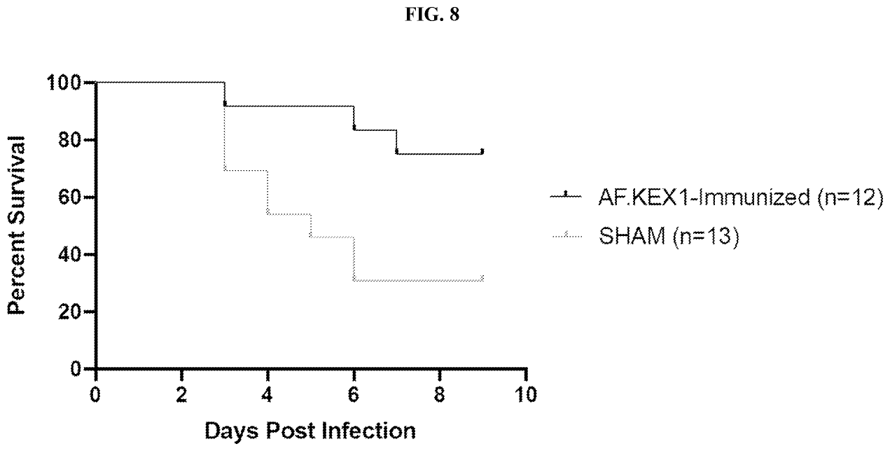

BALB/c mice were immunized with 50 ug AF.KEX1 peptide (n=12) prepared with TITERMAX adjuvant (1:1) or were sham-immunized (n=13). The immunosuppressive regimen was initiated as described by Herbst et al. (Dis Model Mech. 2013; 6(3):643-651. doi:10.1242/dmm.010330), beginning 28 days following immunization. Briefly, mice received 1 mg/kg FK506 (Tacrolimus) intraperitoneally daily and 125 mg/kg hydrocortisone subcutaneously every three days. This immunosuppression regimen began three days prior to Aspergillus challenge and continued through the post-infection observation period (10 days post-infection). Mice were intranasally challenged with 5×10⁶A. fumigatus (strain 293) conidia and monitored twice daily for signs of aspergillosis, (e.g., weight loss, temperature) for 10 days. Nine (9) out of thirteen (13) sham-immunized mice developed aspergillosis compared to three (3) out of the twelve (12) mice that had been immunized with the AF.KEX1 peptide (p=0.0183), (FIG. 8). The results demonstrated that the AF-KEX1 immunogen was protective against the development of aspergillosis and was effective as a vaccine immunogen in immunosuppressed animals treated with the peptide.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1-25. (canceled)

26. A method of treating or protecting an immunosuppressed patient against developing aspergillosis and/or the symptoms thereof associated with infection by an Aspergillus fungal pathogen, the method comprising:
administering to a patient who is to receive, is receiving, or has received an immune suppressive drug or medication a Kexin peptide derived from an Aspergillus fungal pathogen in an amount effective for the patient to generate anti Aspergillus Kexin peptide antibodies and acquire protective immunity to treat or protect the immunosuppressed patient against developing aspergillosis and/or the symptoms thereof.

27. A method of treating or protecting an immunosuppressed patient against developing aspergillosis and/or the symptoms thereof associated with infection by an Aspergillus fungal pathogen, the method comprising:
administering to a patient who is to receive, is receiving, or has received an immune suppressive drug or medication an isolated antiserum comprising an antibody produced against the Aspergillus Kexin peptide, or an isolated and purified antibody produced against the Aspergillus Kexin peptide, in an amount effective for the patient to acquire protective immunity to treat or protect the immunosuppressed patient against developing aspergillosis and/or the symptoms thereof.

28. The method of claim 26, wherein the Kexin peptide immunogen derived from an Aspergillus fumigatus fungal pathogen is administered to the patient.

29. The method of claim 26, wherein the patient has congenital or acquired immunosuppression, is undergoing treatment with an immunosuppressive drug, agent, or medicament, is undergoing treatment with an anticancer, chemotherapeutic, anti-inflammation or immuno-oncology drug, agent, or medicament, or is a pre-transplant patient or a post-transplant patient.

30. The method of claim 26, wherein the patient is to receive, is receiving, or has received one or more immunosuppressive drugs or agents.

31. The method of claim 26, wherein the Aspergillus fungal pathogen is selected from Aspergillus fumigatus (A. fumigatus), Aspergillus flavus (A. flavus), Aspergillus terreus (A. terreus), Aspergillus nidulans (A. nidulans), Aspergillus versicolor (A. versicolor), or Aspergillus niger (A. niger).

32. The method of claim 31, wherein the Aspergillus fungal pathogen is Aspergillus fumigatus (A. fumigatus).

33. The method of claim 26, wherein the aspergillosis is selected from allergic bronchopulmonary aspergillosis (ABPA), allergic Aspergillus sinusitis, aspergilloma, chronic pulmonary aspergillosis, invasive pulmonary aspergillosis (IPA), or cutaneous aspergillosis.

34. The method of claim 33, wherein the aspergillosis is invasive pulmonary aspergillosis (IPA).

35. A method of treating or protecting a subject from aspergillosis and/or the symptoms thereof associated with infection by an Aspergillus fungal pathogen, the method comprising: administering to a subject in need thereof an isolated antiserum comprising an antibody directed against an Aspergillus Kexin peptide immunogen, or an isolated and purified antibody directed against the Aspergillus Kexin peptide immunogen, in an amount effective to treat or protect the subject from aspergillosis and/or the symptoms thereof.

36. (canceled)

37. The method of claim 35, wherein the antiserum or the antibody is generated against a 90-amino acid Kexin peptide comprising or consisting of SEQ ID NO: 2, or an 88-amino acid Kexin peptide comprising or consisting of SEQ ID NO: 3.

38-51. (canceled)

52. A kit comprising an Aspergillus Kexin peptide of SEQ ID NO: 2 or an Aspergillus Kexin peptide of SEQ ID NO: 3, an expression vector comprising a polynucleotide encoding the Aspergillus Kexin peptide, or an isolated antiserum comprising antibodies specifically directed against Aspergillus Kexin peptide for use in the method of claim 1.

53. (canceled)

54. A method of treating, protecting, or reducing the severity of aspergillosis disease and/or the symptoms thereof in a subject, the method comprising: administering to a subject in need thereof an effective amount of a Kexin peptide derived from an Aspergillus fungal pathogen to treat aspergillosis disease and/or the symptoms thereof in the subject.

55. The method of claim 54, wherein the Aspergillus Kexin peptide is selected from a 90-amino acid peptide comprising or consisting of SEQ ID NO: 2 or an 88-amino acid peptide comprising or consisting of SEQ ID NO: 3.

56. The method of claim 54, wherein the Aspergillus fungal pathogen is selected from Aspergillus fumigatus (A. fumigatus), Aspergillus flavus (A. flavus), Aspergillus terreus (A. terreus), Aspergillus nidulans (A. nidulans), Aspergillus versicolor (A. versicolor), or Aspergillus niger (A. niger).

57. The method of claim 54, wherein the Aspergillus fungal pathogen is Aspergillus fumigatus (A. fumigatus).

58. The method of claim 54, wherein the aspergillosis disease is selected from allergic bronchopulmonary aspergillosis (ABPA), allergic Aspergillus sinusitis, aspergilloma, chronic pulmonary aspergillosis, invasive pulmonary aspergillosis (IPA), or cutaneous aspergillosis.

59. The method of claim 9, wherein the aspergillosis disease is invasive pulmonary aspergillosis (IPA).

60. The method of claim 54, wherein the subject is immunosuppressed or immunocompromised.

61. A method of preventing or reducing the development of aspergillosis associated with infection by an Aspergillus fungal organism, the method comprising:
administering to a subject in need thereof an Aspergillus Kexin peptide in an amount effective to elicit an immune response comprising Aspergillus Kexin peptide-specific antibodies in the subject, wherein the anti-Aspergillus Kexin peptide antibodies prevent or reduce the development of aspergillosis associated with infection by the Aspergillus fungal organism in the subject.

62. The method of claim 54, wherein the method reduces lung fungal burden associated with infection by an Aspergillus fungal organism.

63. The method claim 54, wherein the method elicits a humoral immune response that is immunoprotective against aspergillosis and/or the symptoms thereof.


SEQUENCE LISTING (3 sequences)

SEQ ID NO: 1 - protein, 844 aa, Aspergillus fumigatus
MRFLGSIALVLSSISVASANVRSRSYDTHEFFALHLDDSASPSHVAQLLGARHEGQIGELANHHTFSIPRERSSDLDALLERARAARKIRRRARDDATSQEQHNDALGGILWSQKLAPKKRLVKRVPPPERLARTFATGKEDPVAAQSQKRIASTLGITDPIFNGQWHLFNTVQLGHDLNVTGVWMEGITGKGVTTAVVDDGLDMYSNDLKPNYFPEGSYDFNDHTPEPRPRLSDDKHGTRCAGEIAAARNDVCGVGVAYDSRVAGVRILSKAIDDADEATAINFAYQENDIFSCSWGPPDDGATMEGPGILIKRAFVNGVQNGRGGKGSIFVFAAGNGASFEDNCNFDGYTNSIYSITVGAIDREGNHPSYSESCSAQLVVAYSSGSGDAIHTTDVGTDKCYSFHGGTSAAGPLAAGTVALALSARPELTWRDAQYLMVETAVPIHEDDGSWQVTKAGRKFSHDWGYGKVDAYALVQKAKTWELVKPQAWFHSPWLRVQHKVPQGDQGLASSYEVTEQMMKNANIARLEHVTVTMNVNHTRRGDLSVELRSPEGIVSHLSTTRKSDNEKAGYVDWTFMTVAHWGESGVGRWTVIVKDTNVNEFTGEFIDWRLNLWGEAIDGANQKPHPFPDEHDDDHSIEDAIVATTSVETGPTKTGVPGSTDDTINRPVNAKPVETQTPSPAETTATKLAPPAETRPAATATSSPTPPAASDSFLPSFMPTFGASKRTQIWIYAAIGSIIVFCIGLGIYFQVQRRKRILNNPRDDYDFEMIEDENALHGGNGRSGRTQRRGGELYNAFAGESDEEEPLFSDEDDEPYRDRAPSEDRLRDTSSDDRSLRHGDH

SEQ ID NO: 2 - protein, 90 aa, Aspergillus fumigatus
PDDGATMEGPGILIKRAFVNGVQNGRGGKGSIFVFAAGNGASFEDNCNFDGYTNSIYSITVGAIDREGNHPSYSESCSAQLVVAYSSGSG

SEQ ID NO: 3 - protein, 88 aa, Aspergillus fumigatus
DDGATMEGPGILIKRAFVNGVQNGRGGKGSIFVFAAGNGASFEDNCNFDGYTNSIYSITVGAIDREGNHPSYSESCSAQLVVAYSSGS